SEQUENCE LISTING (27 sequences)

SEQ ID NO: 1 - protein, 17 aa, artificial sequence
  synthetic peptide
KRRVVQREKRAVGIGAL

SEQ ID NO: 2 - protein, 17 aa, artificial sequence
  synthetic peptide
KRRVVQREKSAVGIGAL

SEQ ID NO: 3 - protein, 17 aa, artificial sequence
  synthetic peptide
KRRVVQREKRAVGIGAL

SEQ ID NO: 4 - DNA, 41 bp, artificial sequence
  synthetic oligonucleotide, wherein N can be any nucleotide
ccaccatgnnnnnnnnnnnnnnnnnnnnnnnnnnnnnntga

SEQ ID NO: 5 - DNA, 41 bp, artificial sequence
  synthetic polynucleotide, wherein N can be any nucleotide
ggtggtacnnnnnnnnnnnnnnnnnnnnnnnnnnnnnnact

SEQ ID NO: 6 - protein, 4 aa, artificial sequence
  synthetic polypeptide
GGGS

SEQ ID NO: 7 - protein, 4 aa, artificial sequence
  synthetic polypeptide
KDEL

SEQ ID NO: 8 - protein, 20 aa, artificial sequence
  synthetic polypeptide
CTKTRHSRRSRRSLTVQTHG

SEQ ID NO: 9 - protein, 20 aa, artificial sequence
  synthetic polypeptide
CTTTGEHRREKRSVALVPHV

SEQ ID NO: 10 - protein, 19 aa, artificial sequence
  synthetic polypeptide
CTTTGEHRREKSVALVPHV

SEQ ID NO: 11 - protein, 17 aa, artificial sequence
  synthetic polypeptide
KRRVVQREKRAVGIGAL

SEQ ID NO: 12 - protein, 17 aa, artificial sequence
  synthetic polypeptide
KRRVVQREKSAVGIGAL

SEQ ID NO: 13 - protein, 20 aa, artificial sequence
  synthetic polypeptide
PTKAKRRVVQREKRAVGIGA

SEQ ID NO: 14 - protein, 20 aa, artificial sequence
  synthetic polypeptide
PVKRYSSAPVRNKRGVFVLG

SEQ ID NO: 15 - protein, 20 aa, artificial sequence
  synthetic polypeptide
GTCTTTGEHRREKRSVALVP

SEQ ID NO: 16 - protein, 20 aa, artificial sequence
  synthetic polypeptide
ATGPRNVPQRRKKRGLFGAK

SEQ ID NO: 17 - protein, 18 aa, artificial sequence
  synthetic polypeptide
MRHKRSAKRTKRASATQL

SEQ ID NO: 18 - protein, 20 aa, artificial sequence
  synthetic polypeptide
GVAGLITGGRRTRREAIVNA

SEQ ID NO: 19 - protein, 20 aa, artificial sequence
  synthetic polypeptide
LILNCHHHASRVARMASDEF

SEQ ID NO: 20 - protein, 20 aa, artificial sequence
  synthetic polypeptide
PELKQKSSNSRKKRSTSAGP

SEQ ID NO: 21 - protein, 19 aa, artificial sequence
  synthetic polypeptide
YMAQACAGNRVRRSVGSSL

SEQ ID NO: 22 - protein, 20 aa, artificial sequence
  synthetic polypeptide
HLPLETFTRHRQPRGWEQLE

SEQ ID NO: 23 - protein, 20 aa, artificial sequence
  synthetic polypeptide
SGLGGAPLGLRLPRETDEEP

SEQ ID NO: 24 - protein, 20 aa, artificial sequence
  synthetic polypeptide
DLDQNTIETMRKPRCGNPDV

SEQ ID NO: 25 - protein, 20 aa, artificial sequence
  synthetic polypeptide
LKPAKSARSVRAQRHTDMPK

SEQ ID NO: 26 - protein, 20 aa, artificial sequence
  synthetic polypeptide
YRLESQQTNRRKKRALDAAY

SEQ ID NO: 27 - protein, 20 aa, artificial sequence
  synthetic polypeptide
KLDVYRQVHSIIRRSLPATL

(19) United States
(12) Patent Application Publication
(10) Pub. No.: US 2017/0166950 A1
(43) Pub. Date: Jun. 15, 2017

(54) COMPOSITIONS AND METHODS FOR IDENTIFYING ENZYME MODULATORS OR INHIBITORS

(71) Applicant: SAN DIEGO STATE UNIVERSITY (SDSU) FOUNDATION, San Diego, CA (US)

(72) Inventor: Roland WOLKOWICZ, San Diego, CA (US)

(73) Assignee: SAN DIEGO STATE UNIVERSITY (SDSU) FOUNDATION, San Diego, CA (US)

(21) Appl. No.: 14/760,456

(22) PCT Filed: Jan. 18, 2014
(86) PCT No.: PCT/US2014/012148

§ 371 Date: Jul. 10, 2015

Related U.S. Application Data
(60) Provisional application No. 61/754,573, filed on Jan. 19, 2013.

(51) Int. Cl. C12Q 1/37; C07K 14/575; C07K 14/705

(52) U.S. Cl. CPC C12Q 1/37; C07K 14/70517; C07K 14/57554; G01N 2500/02; G01N 2500/10; C07K 2319/03; C07K 2319/02; C07K 2319/50; C07K 2319/43; C07K 2319/035; C07K 2319/42

(57) ABSTRACT

The invention is directed to compositions to screen for compounds, e.g., small molecules or drugs, that can modulate or inhibit enzymes, e.g., proteases, such as viral proteases, e.g., HIV proteases; and methods for making and using these compositions. In alternative embodiment, the invention provides compositions and methods for identifying compositions, e.g., drug molecules that can modulate or inhibit enzymes, e.g., proteases, proteinases or peptidases or the like, e.g., HIV proteases. In alternative embodiments, the invention provides cell-based assays to screen for compositions, e.g., small molecules or drugs, that modulate or inhibit or modify the activity of enzymes such as proteases, proteinases or peptidases or the like, such as calcium-dependent protein convertases involved in HIV envelop protein processing, including cleavage of the HIV gp160 envelope precursor, resulting in gp120 and gp41 envelope products. In alternative embodiment, the compositions and methods of the invention are adapted for high through-put or multiplexed screening of compounds, e.g., drug molecules that can modulate or inhibit enzymes.

### TECHNICAL FIELD

This invention relates to molecular and cellular biology, biochemistry, molecular genetics, and drug design and discovery. In one aspect, the invention is directed to compositions to screen for compounds such as small molecule drugs that inhibit, act as competitor, or modulate an enzyme such as a protease, e.g., a protease, proteinase or peptidase or the like, e.g., viral proteases, e.g., an HIV-1 protease. In alternative embodiment, the compositions and methods of the invention are adapted for high through-put or multiplexed screening of compounds, e.g., drug molecules that can modulate or inhibit enzymes.

### BACKGROUND

The human immunodeficiency virus (HIV), identified as the causative agent of AIDS in 1981, has resulted in over 33 million deaths since then. Three decades of HIV research have resulted in antivirals targeting the viral proteins necessary for HIV infection, mainly Protease, Reverse Transcriptase, and recently, Integrase and Envelope (Env). In total 32 inhibitors have been approved by the FDA since Saquinavir, the first Protease inhibitor, in 1995. Inhibitors supplied as a cocktail of three or more inhibitors in the form of Highly Active Anti-retroviral Therapy (HAART) have resulted in a drastic reduction in the number of AIDS-related deaths. Despite the significant progress achieved with the development of HAART, AIDS still remains a devastating disease. Emergence of resistant strains together with the terrible side-effects of existing drugs, and lack of success with vaccine development, highlights the need for novel antivirals as well as innovative methods to facilitate their discovery.

### SUMMARY

The invention provides compositions, assays and cell-based methods for monitoring the activity of an enzyme, e.g., a protease, proteinase or peptidase or the like, e.g., a viral protease or an HIV-1 protease (PR). The invention provides compositions, assays and cell-based methods for detecting or assaying for modulators (e.g., inhibitors or competitors, or activators) of the activity of an enzyme, e.g., a protease, proteinase or peptidase or the like, e.g., a viral protease or an HIV-1 protease (PR). Thus, in one embodiment, invention provides compositions, assays and cell-based methods for drug discovery.

In alternative embodiments, the invention provides chimeric or recombinant scaffold proteins comprising:
(a)
   (i) an amino acid motif or subsequence susceptible to cleavage (“a cleavable target motif”) by an enzyme or a protease under physiologic or cell culture conditions,
   and optionally the enzyme or protease is a viral, a bacterial, an Archaeal, a eukaryotic, a mammalian, a human, an HIV or an HIV-1 enzyme or protease, or a furin,
   and optionally the cleavable target motif is a viral, a bacterial, an Archaeal, a eukaryotic, a mammalian, or a human sequence (or target, or substrate), optionally within an HIV-1 envelope (Env) protein, and optionally the cleavable target motif is cleaved by a furin or a protein convertase,
   and optionally the cleavable target motif is at the gp120/gp41 boundary within a gp160 Env poly-protein;
   (ii) a transmembrane domain;
   (iii) a signal sequence or any amino acid motif that places the scaffold protein on the extracellular surface of the cell, or a signal sequence for scaffold protein insertion into the Endoplasmic Reticulum (ER) for transport to the cell surface,
   wherein optionally the signal sequence is a prolactin signal sequence; and
   (iv) at least two detectable moieties,
   wherein optionally one of the detectable moieties is a FLAG tag or an epitope for an antibody, and optionally the FLAG tag is positioned distal to the cleavable target motif (with respect to the cell when the scaffold protein is on the extracellular surface of the cell),
   and optionally one of the detectable moieties is a Human influenza hemagglutinin (HA) tag, and optionally the HA tag is positioned on the scaffold protein between the cleavable target motif and the extracellular surface of the cell when the scaffold protein is on the extracellular surface of the cell,

wherein: when the scaffold protein is expressed on the cell surface the amino acid motif or subsequence susceptible to cleavage by the enzyme or protease (“the cleavable target motif”) is positioned within the scaffold protein such that at least one of the detectable moieties is distal to the cleavable target motif (with respect to the cell), and at least one of the detectable moieties is between the cleavable target motif and the cell surface when the scaffold protein is expressed extracellularly,

thus, if the enzyme or protease is active in the cell, the detectable moiety or moieties distal to the cleavable target motif will not be present (or part of) the scaffold protein expressed on the cell surface, but when an effective amount of an inhibitor or a competitor to the enzyme or protease is present in the cell, the cleavable target motif is not cleaved and the detectable moiety or moieties distal to the cleavable target motif will be present (or part of) the scaffold protein expressed on the cell surface;

(b) the chimeric or recombinant scaffold protein of (1), wherein the scaffold protein comprises all of, substantially all of, or part of: a murine CD8a homolog or a Lyt2 transmembrane domain glycoprotein receptor polypeptide as the scaffold;

(c) the chimeric or recombinant scaffold protein of (a) or (b), wherein the enzyme or protease is a viral enzyme or protease, or an HIV enzyme or protease, or a furin, or a proteinase or peptidase; or

(d) the chimeric or recombinant scaffold protein of any of (a) to (c), wherein the amino acid motif or subsequence susceptible to cleavage (“cleavable target motif”) is from or is derived from a virus of the family Flaviviridae, or a Dengue virus, a Hepatitis C Virus, a West Nile virus, a Yellow fever virus, a Japanese encephalitis virus, a Tick-borne encephalitis virus, a Kyasanur Forest disease virus, a Murray Valley encephalitis virus, a St. Louis encephalitis virus, a bovine viral diarrhoea virus, a Rio Bravo virus, a Culex flavivirus or pegivirus, an influenza virus, a papilloma virus, a Sindbis virus and/or an Ebola virus; or, an amino acid motif or subsequence susceptible to cleavage as illustrated in FIG. 20.

In alternative embodiments, the invention provides isolated, recombinant or synthetic nucleic acids encoding a scaffold protein of the invention,

wherein optionally the nucleic acid is operatively linked to a transcriptional regulatory unit, and optionally the transcriptional regulatory unit comprises a promoter, and optionally the promoter is a constitutive or an inducible promoter,

and optionally the nucleic acid further comprises a sequence encoding the enzyme or protease, and optionally the enzyme or protease-coding sequence is operatively linked to the same or a similar transcriptional regulatory unit as the nucleic acid encoding the scaffold protein of the invention.

In alternative embodiments, the invention provides a vector, expression cassette, cosmid or plasmid comprising the isolated, recombinant or synthetic nucleic acid of the invention.

In alternative embodiments, the invention provides a cell comprising:

(a) the chimeric or recombinant scaffold protein of the invention; the isolated, recombinant or synthetic nucleic acid of the invention; and/or, the vector, expression cassette, cosmid or plasmid of the invention;

(b) the cell of (a), wherein the enzyme or protease is heterologous to the cell and the cell further comprises a heterologous nucleic acid encoding the heterologous enzyme or protease,

and optionally the enzyme or protease-coding sequence is operatively linked to the same or a similar transcriptional regulatory unit as the nucleic acid encoding the scaffold protein of the invention,

and optionally the enzyme or protease-coding sequence is contained in or is part of the same or a different vector, expression cassette, cosmid or plasmid of the invention;

(c) the cell of (a) or (b), wherein the cell constitutively or inducibly expresses the chimeric or recombinant scaffold protein of the invention; or

(d) the cell any of (a) to (c), wherein the cell is a mammalian cell, a monkey cell, or a human cell, or a lymphocyte or a T-cell.

In alternative embodiments, the invention provides a cell line, or a stable cell line, derived from the cell of the invention.

In alternative embodiments, the invention provides a non-human transgenic animal comprising: the chimeric or recombinant scaffold protein of the invention; the isolated, recombinant or synthetic nucleic acid of the invention; a cell line or a stable cell line of the invention; and/or, the vector, expression cassette, cosmid or plasmid of the invention.

In alternative embodiments, the invention provides a cell-based method for monitoring the activity of an enzyme, a protease, a viral protease, or an HIV-1 protease (PR), comprising:

(a) (i) providing a cell of the invention, or a cell line of the invention, or non-human transgenic animal of the invention, wherein the cell or cell line expresses the chimeric or recombinant scaffold protein of the invention,

wherein optionally the enzyme or protease is endogenous to the cell or is heterologous to the cell; and

(b) determining whether the detectable moiety or moieties distal to the cleavable target motif on the scaffold protein is expressed on the scaffold protein on the extracellular surface of the cell, or whether or not the detectable moiety or moieties distal to the cleavable target motif on the scaffold protein are or are not be present (or part of) the scaffold protein expressed on the cell surface,

wherein lack of detection of the detectable moiety or moieties distal to the cleavable target motif on the scaffold protein indicates that the enzyme or protease is active in the cell, and detection of the detectable moiety or moieties distal to the cleavable target motif on the scaffold protein indicates the presence of an inhibitor or a competitor of the enzyme or protease,

wherein extracellular detection of the detectable moiety or moieties positioned on the scaffold protein between the cleavable target motif and the cell surface when the scaffold protein is expressed on the cell surface indicates or confirms that the scaffold protein is expressed on the cell surface.

In alternative embodiments, methods of the invention further comprise screening for a putative inhibitor or competitor of an enzyme, a protease, a viral protease, an HIV protease, or an HIV-1 protease, by:

(a) providing a compound to be screened as an inhibitor or a competitor of an enzyme, a protease, a viral protease, an HIV protease, or an HIV-1 protease; or a nucleic acid encoding a protein to be screened as an inhibitor or a competitor of an enzyme, a protease, a viral protease, an HIV protease, or an HIV-1 protease;

(b) contacting a plurality of the cells with the compound or nucleic acid of (a),

wherein optionally the contacting is either before, during and/or after expression of the scaffold protein-expressing nucleic acid in the cell,

and optionally the nucleic acid encoding a protein to be screened as an inhibitor or a competitor of an enzyme, a protease, a viral protease, an HIV protease, or an HIV-1 protease is expressed before, during and/or after expression of the scaffold protein-expressing nucleic acid in the cell; and

(c) determining whether the detectable moiety or moieties distal to the cleavable target motif on the scaffold protein is expressed on the scaffold protein on the extracellular surface of the cell, or whether or not the detectable moiety or moieties distal to the cleavable target motif on the scaffold protein are or are not be present (or part of) the scaffold protein expressed on the cell surface,

wherein lack of detection of the detectable moiety or moieties distal to the cleavable target motif on the scaffold protein indicates that the enzyme or protease is active in the cell, and detection of the detectable moiety or moieties distal to the cleavable target motif on the scaffold protein indicates that the compound is acting as an inhibitor or a competitor of the enzyme or protease (assuming that the same cells cultured under or exposed to the same conditions but not exposed to or contacted with or expressing the putative inhibitor or competitor do not express the detectable moiety or moieties distal to the cleavable target motif on the scaffold protein),

wherein extracellular detection of the detectable moiety or moieties positioned on the scaffold protein between the cleavable target motif and the cell surface when the scaffold protein is expressed on the cell surface indicates or confirms that the scaffold protein is expressed on the cell surface.

In alternative embodiments, methods of the invention further comprise running a negative control comprising dividing the plurality of the cells and not adding the compound to be screened as an inhibitor or competitor to one of the divided cell samples, or not expressing the putative inhibitor or competitor in one of the divided cell samples.

In alternative embodiments, methods of the invention further comprise running a positive control comprising dividing the plurality of the cells and adding a compound known to be an inhibitor or competitor of the enzyme or protease to one of the divided cell samples, or expressing a known inhibitor or competitor of the enzyme or protease in one of the divided cell samples.

In alternative embodiments, detectable moiety is detected or measured on the extracellular surface of the cell by a high throughput screen (HTS), a flow cytometry or a microscope visualization.

In alternative embodiments, the compound to be screened as an inhibitor of the enzyme, protease, viral protease or HIV-1 protease comprises a small molecule, a nucleic acid, a polypeptide or peptide, a peptidomimetic, a polysaccharide or a lipid.

In alternative embodiments, the compound to be screened as an inhibitor of the enzyme, protease, viral protease or HIV-1 protease is a member of a library of compounds to be screened, or a member of a random peptide library or a chemical compound.

In alternative embodiments, the compound to be screened comprises a plurality of compound comprising or from a combination of: retroviral random peptide libraries; combinatorial compound libraries; and/or, endogenously expressed random peptide libraries specifically targeted to the compartment where Env processing occurs.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 schematically illustrates an exemplary cell-based drug discovery assay strategy of the invention; as discussed in detail in Example 1, below.

FIG. 2 schematically illustrates a gp160 Env precursor, which in alternative embodiments is incorporated into an exemplary scaffold protein of the invention; as discussed in detail in Example 1, below.

FIG. 3 schematically illustrates an exemplary assay of the invention comprising a scaffold protein containing two tags (HA and FLAG) separated by the gp120/gp41 Furin recognition/cleavage site, is targeted to the ER; as discussed in detail in Example 1, below.

FIG. 4 schematically illustrates exemplary constructs of the invention, or constructs that can be used to practice methods of the invention, including an exemplary engineered scaffold with a minimal gp120/gp41 boundary, including a pBMN-gp160 min-wt (SEQ ID NO:1) and pBMN-gp160 min-mut (SEQ ID NO:2); as discussed in detail in Example 1, below.

FIG. 5 illustrates graphically and schematically: transient expression in 293T cells of the pBMN-gp160 min-wt (SEQ ID NO:1)-comprising and pBMN-gp160 min-mut (SEQ ID NO:2)-comprising constructs; and, graphically illustrate flow cytometry data from experiments using naive cells (top panels), or infected with wt gp120/gp41 boundary (mid panels) or mutant boundary (bottom panels) constructs, which were analyzed by flow cytometry post-transfection); as discussed in detail in Example 1, below.

FIG. 6 illustrates graphically and schematically: transient expression in 293T cells of the pBMN-gp160 min-wt (SEQ ID NO:1)-comprising and pBMN-gp160 min-mut (SEQ ID NO:2)-comprising constructs; and, graphically illustrates a flow cytometry analysis of SupT1 clones stably expressing the assay; and naïve cells or cells expressing the wt or the mutant version of the boundary were analyzed following staining with both APC-coupled anti-HA and FITC-coupled anti FLAG antibodies); as discussed in detail in Example 1, below.

FIG. 7 illustrates graphically and schematically: SupT1 clones stably expressing the assay (as with FIGS. 5 and 6, above, using the pBMN-gp160 min-wt (SEQ ID NO:1)-comprising and pBMN-gp160 min-mut (SEQ ID NO:2)-comprising constructs) following inhibition with DCK; clones were treated with increasing concentration of DCK and stained with FITC-coupled anti FLAG antibodies; naïve cells were used as control for antibody staining); as discussed in detail in Example 1, below.

FIG. 8 illustrates: Fluorescence microscopy images of SupT1 clones stably expressing the assay (as with FIGS. 5, 6 and 7, above, using the pBMN-gp160 min-wt (SEQ ID NO:1)-comprising and pBMN-gp160 min-mut (SEQ ID NO:2)-comprising constructs); clones expressing wild type (wt) or mutant version of the boundary were analyzed with HA-FITC and FLAG-CY3); as discussed in detail in Example 1, below.

FIG. 9A illustrates schematically: exemplary retroviral/lentiviral constructs for inducible expression: the rtTA coupled to mCherry (the so called exemplary pBMN-rtTA construct) and the TRE coupled to a minimal CMV promoter driving GFP (the so called exemplary pTRE-GFP construct); and, FIG. 9B illustrates schematically and graphically results from studies using these constructs: left panels illustrate fluorescence microscopy images showing activation of GFP only in the presence of Doxycycline (Dox); and right panels graphically represent these images by flow cytometry; as discussed in detail in Example 1, below.

FIGS. 10A, 10B, 10C, 10D, 10E, 10F, 10G, 10H, 10I and 10J schematically illustrate exemplary constructs of the invention; as discussed in detail in Example 1, below.

FIGS. 11a and 11b schematically illustrate exemplary constructs of the invention; in particular, constructs for the expression of the random peptide libraries; scaffolds for: FIG. 11a. ER-TGN-retained peptides (the “ER-TGN retained library”), and, FIG. 11b. secreted peptides (the “secreted library”); as discussed in detail in Example 1, below.

FIG. 12 illustrates an exemplary process of the invention for the construction of an exemplary peptide library, and also illustrates exemplary constructs of the invention; as discussed in detail in Example 1, below.

FIG. 13 schematically illustrates an exemplary analysis scheme of the invention, e.g., where putative rescued peptides can be transferred into assay-bearing naïve cells to corroborate inhibition of cleavage and thus FLAG retention; as discussed in detail in Example 1, below.

FIG. 14 schematically illustrates an analysis of an exemplary assay of the invention in a 96-well format; as discussed in detail in Example 1, below.

As example, FIG. 15 schematically illustrates a fluorescent flow cytometry analysis of three SupT1 T cell lines, each carrying a different substrate within the context of the assay elements; as discussed in detail in Example 1, below.

FIG. 16 schematically illustrates a fluorescent flow cytometry analysis in the PE channel for td Tomato expression and FITC channel for FLAG expression; as discussed in detail in Example 1, below.

FIG. 17 graphically illustrates experiment data demonstrating that inducible short interfering RNA expression against Furin corroborates the results obtained and shown in FIG. 16; as discussed in detail in Example 1, below.

FIG. 18 graphically illustrates that knockdown of Furin reconstitutes FLAG expression with wildtype HIV-1 envelope boundary for a period of at least five days, while no reconstitution is observed with DenV-prM for the same period; as discussed in detail in Example 1, below.

FIG. 19 schematically illustrates flow cytometry data demonstrating that the pattern of cleavage is different in: HIV wild type and mutant boundaries; wild type DenV pr-M boundaries; mutant DenV pr-M boundaries; and control and a pr-M boundary of West Nile virus; as discussed in detail in Example 1, below.

FIG. 20 is a table listing alternative “targets” or “substrates” that can be incorporated in exemplary constructs and assays of the invention; as discussed in detail in Example 1, below.

Like reference symbols in the various drawings indicate like elements.

### DETAILED DESCRIPTION

The invention provides methods and compositions, including chimeric recombinant proteins, nucleic acids that encode them, and cells and kits comprising them, to screen for compositions, e.g., small molecule drugs, that can modulate, e.g., inhibit, compete or modulate, e.g., enhance, enzymes, e.g., proteases, proteinases or peptidases or the like, e.g., viral proteases, including retroviral (e.g., HIV) proteases. In alternative embodiments, the invention provides cells and cell-based assays for monitoring the activity of enzymes, e.g., proteases, proteinases or peptidases or the like, e.g., viral proteases, e.g., HIV-1 protease (PR), which is an aspartyl protease. In one embodiment, these cells and cell-based assays are used to screen for and identify novel viral protease, e.g., PR, inhibitors or modulators, such as competitors. In one embodiment, assays of the invention effectively couple the surface (extracellular) expression of a protein used as a scaffold (a scaffold protein), and expression of detectable markers, or “tags” on the scaffold, with the activity of the enzymes, e.g., proteases, proteinases or peptidases or the like, e.g., viral protease, e.g., PR. In alternative embodiments, the compositions (e.g., scaffolds), the methods (e.g., assays for drug discovery) and cells of the invention comprise use of at least two markers on the extracellularly expressed scaffold, i.e., the scaffold is “double-tagged”, wherein an cleavable target, e.g., a protease target, is placed on the scaffold between at least two of the tags, and when an effective amount of an inhibitor or a competitor of the enzyme (e.g., a protease, such as a furin) is present in the cell, the enzyme will not cleave the target, and the tag positioned distal (from the cell) on the scaffold (e.g., a FLAG tag) is not released, and only at least a second tag (positioned between the cleavable target and the cell surface) remains on the cell surface. Alternatively, if the enzyme (e.g., protease) is not inhibited, the target is cleaved and the distal tag will not remain on the scaffold or be associated with the cell's surface. Thus, in these embodiments, drug discovery assays screen for tag-positive FLAG positive cells, e.g., by FACS or equivalents, to screen for putative drugs, i.e., the inhibitors or competitors of the enzymes or proteases.

In alternative embodiments, compositions and assays of the invention, scaffolds of the invention comprise use of at least recognizable or detectable two tags expressed as a part of the scaffold that utilizes the classical secretory pathway for transport into the cell surface. In alternative embodiments, compositions and assays of the invention can use any recognizable or detectable marker, or “tag”. One exemplary composition and assay of the invention, as described in the Examples, is based on two independent TAGs; HA and FLAG. However, compositions and assays of the invention are adaptable to any recognizable or detectable marker, e.g., tag or fluorescent protein, of choice. The tags, separated by a putative cleavable site within the ER/Golgi/TGN, can be any tag of choice, provided they are recognizable or detectable. In alternative embodiments, they can be any antigen or TAGs, e.g., when antibodies that recognize them are available, or they can be a fluorescent compound or proteins.

In alternative embodiments, compositions and assays of the invention can detect a modulator, e.g., inhibitor or activator, of any enzyme, e.g., proteases, proteinases or peptidases or the like. One exemplary composition and assay of the invention, as described in the Examples, is based on a scaffold with two tags, separated by a putative cleavable site within the ER/Golgi/TGN compartment. While the data described in the Example shows a cleavable site specifically cleaved by Furin and other protein convertases (PCs), alternative embodiments of compositions and/or assays of the invention can be designed as a platform for any putative or known cleavable site in the ER/Golgi/TGN compartment. In alternative embodiments, the assay can monitor cleavage by any enzyme, e.g., a protease, proteinase or peptidase or the like, provided the enzyme is expressed in (and can be recombinantly expressed or naturally expressed, or functions and/or resides in the ER/Golgi/TGN compartment.

In alternative embodiments, compositions and assays of the invention can be used to assay for or detect any modulator for any enzyme cleavable target or substrate. The exemplary assay as described in the Examples relies on the cleavage of the HIV envelope gp120/gp41 boundary, chosen as proof of principle. This exemplary target is a well-recognized target of Furin/PCs cleaved in the ER/Golgi/TGN compartment. However, in alternative embodiments, compositions and assays of the invention are adaptable to any target of choice; whether viral or of cellular organisms, provided it can be cleaved in the ER/Golgi/TGN compartment. In alternative embodiments, compositions and assays of the invention provide a platform for the monitoring of cleavage of proteins processed, cleaved and/or matured during the travelling or residency within the ER/Golgi/TGN compartment.

In alternative embodiments, compositions and assays of the invention can be used to study of the cleavage and or transport process. In alternative embodiments, compositions and assays of the invention can be used to monitor the cleavage/maturation process as well as the process of transport to the cell surface. In alternative embodiments, compositions and assays of the invention can be used to study the proteins and/or factors that influence or are required for the processes of protein cleavage, processing and/or maturation in the ER/Golgi/TGN compartment. As the compositions and assays of the invention comprise use of a scaffold protein that travels to the cell surface, they can also be used to learn about the factors and/or proteins required for transport to the cell surface.

In alternative embodiments, compositions and assays of the invention can be used to for drug discovery. In alternative embodiments, compositions and assays of the invention can be used to screen for inhibitors and/or competitors against the process of cleavage of the substrate inserted between the two detectable markers, e.g., flags, and/or against the activity of the enzymes responsible for their cleavage, provided the enzyme is expressed in (and the enzyme can be recombinantly expressed or homologous to the cell of the assay), or reside and/or function in the ER/Golgi/TGN compartment.

In one embodiment, the scaffold is engineered for its conditional expression on the surface of a cell, e.g., a eukaryotic, a yeast or a mammalian cell. For that purpose, in one embodiment, the scaffold is fused to a signal sequence to enable efficient and/or directed transport, and a transmembrane domain (e.g., an Lyt2, the murine CD8 molecule, and the like) is used to enable subsequent insertion in the cell membrane. In one embodiment, at least two detectable markers, e.g., “tag”, including a FLAG tag, is added to the scaffold downstream of the signal sequence for detection, e.g., for antibody detection, e.g., through flow cytometry or equivalent visualization.

In one embodiment, the assay co-expresses both the scaffold protein and the enzyme or protease, e.g., viral protease, e.g., the HIV-1 PR, which if active will bind to and cleave the scaffold at the protease recognition sequence.

In one embodiment, both scaffold and protease are co-expressed in T cells, e.g., SupT1 T-cells, in an inducible off/on-based vector system (e.g., activated upon addition of tetracycline or doxycycline). Inducible expression of protease, e.g., PR, helps avoid its possible cytopathic effects.

In one embodiment, the logic behind the engineering of the scaffold as a membrane-expressed protein is as follows: in the presence of the active viral protease, the proteolytic enzyme will cleave the scaffold, resulting in the loss of transmembrane domain, thus preventing tag cell surface expression. In the absence of protease, or when protease is blocked or inhibited, the scaffold will be intact and incorporated into the membrane. As a result, the surface expression of the scaffold can be determined by flow cytometry allowing the discrimination between active and inactive or blocked protease.

The assay is cell-based, and can be easily implemented for a high throughput screen, e.g., FACS. As such, the assay is invaluable for drug discovery, and can be utilized in biological screens aimed at finding novel enzyme, e.g., protease, inhibitors or modulators through random peptide libraries or chemical compounds libraries.

In one embodiment, the invention provides assays that can be adapted for a high throughput manner using e.g. flow cytometry such as FACS, and can discriminate between active and non-active or blocked protease. In one embodiment, the invention provides assays that can be easily adapted for high throughput screening. In one embodiment, the invention provides assays of this invention can be used to screen for novel enzyme or protease inhibitors or modulators.

In one embodiment, the invention provides assays of this invention adapted for the screen of random peptide libraries or chemical compounds for drug discovery.

In one embodiment, the methods of the invention use a random peptide library or any peptide of choice, which can be introduced ‘in cis’, replacing the p2/p7 recognition/cleavage site, enabling the discovery of higher affinity sites for enzyme or protease, e.g., PR, which can be the basis for the development of competitor peptidomimetic drugs. In one embodiment, the random peptide library is expressed ‘in trans’, enabling the discovery of competitors/inhibitors or modulators for enzyme or protease, e.g., PR, which can be the basis for peptidomimetic drugs.

In one embodiment, the non-biased approach of the invention permits the rescue of peptides or chemicals targeted not necessarily to the catalytic site of enzyme or protease, e.g., PR. Thus, the assays of the invention provide for extensive characterization of enzyme or protease or PR, facilitating the elucidation of interactions of enzyme or protease or PR with cellular targets, its mode of action and modulation, in the context of the host cell. Assays of this invention will permit the replacement of PR with PR from different viral strains or clades, or truncated versions of PR, enabling further dissection of PR activity, and study its modulation through co-expression of cellular factors or addition of drugs.

The assays of this invention can be further adapted to proteases of different viruses such as Hepatitis C by just exchanging the recognition/cleavage site segment of the scaffold. The assays of this invention can thus be exploited for the search for protease inhibitors or modulators against any of the known viral pathogens that utilize their own protease/s as part of their lifecycles.

The assays of this invention can be adapted for the search of HIV envelope processing inhibitors or modulators. One of the HIV proteins, envelope, is processed by furin and other cellular convertases. By just exchanging the recognition/cleavage segment of the scaffold with the envelope recognition site, the assay can be further utilized for the finding of envelope processing inhibitors or modulators. This same scaffold is useful for the search of transport inhibitors or modulators, as envelope is transported through the ER, trans-Golgi network in order to be inserted within the cell membrane.

In alternative embodiments, the assays of this invention comprise expression of a scaffold designed for expression in the cytoplasm that is able to be exported into the cell membrane.

In alternative embodiments, assays of this invention comprise expression of both enzyme, e.g., viral enzyme, such as a PR, and a scaffold of the invention, in an off/on system for inducible expression, or alternatively, for constitutive expression.

In alternative embodiments, assays of this invention comprise expression of a protein that is expressed on the surface of the mammalian cell only when not cleaved by a protease, e.g., an HIV protease.

In alternative embodiments, assays of this invention can be adapted for the screen of random peptide libraries or chemical compounds.

In alternative embodiments, assays of this invention can be implemented in mammalian cells and other cells, e.g., yeast or bacterial cells.

In alternative embodiments, methods provide for the construction of the scaffold and its expression on the cell surface. In alternative embodiments, the p2/p7 scaffold has been engineered as described and effectively expressed on the cell surface. In alternative embodiments, the scaffold has been introduced in a retroviral vector.

We utilized a novel cell-based assay that exploits the classical secretory pathway for the elucidation of the processes involved in HIV-1 envelope (Env) maturation. HIV-1 relies on proteases for the processing of its proteome. While the viral protease cleaves most of the recognition sites within the viral proteome, the site within Env is cleaved by the host enzymes Furin and similar protein convertases (PCs) within the Trans-Golgi Network (TGN). Processing at the gp120/gp41 boundary within the gp160 Env poly-protein is necessary for the production of infectious viral particles. In one embodiment, the recognition of gp160 by Furin is a target for drug development against HIV-1.

We describe an assay based on the engineering of a scaffold protein that will place the gp120/gp41 boundary within the lumen of the Trans-Golgi Network (TGN) where it can be recognized and then processed. The well-established topology of the murine CD8a homolog, Lyt2, transmembrane domain glycoprotein receptor, prompted us to choose Lyt2 as the basic scaffold for the assay. In one embodiment, a Lyt2 FLAG-tagged molecule is fused to the prolactin signal sequence to ensure both antibody-based recognition and proper insertion into the Endoplasmic Reticulum (ER) for transport to the cell surface. Additionally, the gp120/gp41 boundary is introduced between the FLAG tag and an HA tag fused to the Lyt2 transmembrane domain, ensuring that the Env segment faces the lumen of the ER/TGN. In this manner, if Furin/PCs recognize and cleave the gp120/gp41 boundary, the FLAG tag will be released. However, if blocked or inhibited, the FLAG tag will remain attached to the HA-tagged scaffold. The double-tagged engineered scaffold will thus allow for the discrimination between cleaved and non-cleaved events based on the cell surface expression of one or two tags, respectively. Results show a drastic reduction of FLAG surface expression with a scaffold containing the wild-type gp120/41 boundary in comparison to its mutant counterpart, proving the utility of FLAG cell-surface expression as a biosensor, e.g., for the activity of Furin.

This exemplary assay, developed in T-cells to provide the natural milieu of HIV-1 infection, can elucidate the still unclear mechanisms of gp160 maturation, which is a target for the inhibition of HIV infection. This will be the first assay of its kind to be developed in a relevant cellular context, facilitating the discovery of drugs specifically inhibiting the recognition/cleavage of Env rather than the activity of cellular enzymes and will thus be aimed at discovering competitors rather than inhibitors of Furin.

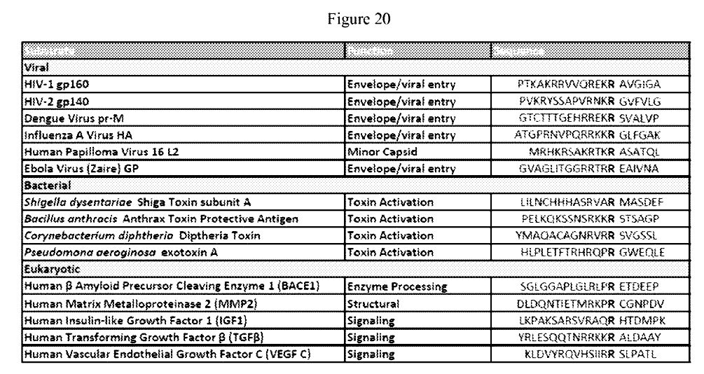

In alternative embodiments, exemplary assays of the invention are adapted to host substrates that utilize the classical secretory pathway. These include targets of the three domains of life, Bacteria, Archaea and Eukarya. These include targets but are not restricted to, enzymes that cleave in the way to the cell surface, at the cell surface, or at the extracellular matrix. These include, but are not restricted to some of the possible substrates, known to be cleaved by Furin and similar enzymes, shown in FIG. 20. In alternative embodiments, targets used in exemplary assays of the invention have important clinical human implications, for example, as in Alzheimer's disease, including substrates for the enzymes: cathepsin, beta-secretase (BACE) (e.g., beta-site APP cleaving enzyme 1), beta- and gamma-secretases, and amyloid precursor protein (APP). In alternative embodiments, targets used in exemplary assays of the invention include furin enzyme substrates, e.g., including those listed in the furin database FurinDB (http://www.nuolan.net/substrates.html), which shows the list of known/putative substrates for Furin. In alternative embodiments, exemplary assays of the invention are adapted to other enzymes that reside and/or are active in the classical secretory pathway.

In alternative embodiments, exemplary assays of the invention are used to monitor other enzymes involved in the classical secretory pathway. In alternative embodiments, exemplary assays of the invention are easily adaptable to monitor enzymes and/or their required factors that reside within the secretory pathway, within the membranes of the secretory pathway, cell surface, or the extracellular matrix. These enzymes/factors include, but are not restricted to protein convertases, peptide peptidases, peptide peptide-peptidases, alpha secretases, beta secretases and gamma secretases. For instance, the amyloid precursor protein (APP) involved in Alzheimer's is cleaved by alpha, beta and gamma secretases.

In alternative embodiments, exemplary assays of the invention are adapted to any detection technique. The assay can be analyzed with any detection technique available, whether it is flow cytometry, microscopy, imaging-based coupled flow cytometry, or any other.

In alternative embodiments, exemplary assays of the invention are used as a platform for drug screening. The utility of the assay, as cell-based, can be adaptable to any screen, including chemical compound libraries, combinatorial libraries, peptide libraries or retrovirally-expressed peptide libraries.

In alternative embodiments, exemplary assays of the invention are used as a platform for target discovery. Targets may include, but not be restricted to: a) enzymes or factors involved in the recognition of the substrate under study (inserted between the two tags), b) enzymes or factors involved in the cleavage of the substrate under study (inserted between the two tags), c) factors involved in the Endoplasmic Reticulum/Golgi/TransGolgi Network, d) factors involved in insertion or targeting to the Endoplasmic Reticulum, e) factors involved in the transport to the cell surface, f) cofactors required for any of the processes mentioned above or any of their combinations.

In alternative embodiments, exemplary assays of the invention are used as a platform for target discovery. Targets may include, but not be restricted to: a) enzymes or factors involved in the recognition of the substrate under study (inserted between the two tags), b) enzymes or factors involved in the cleavage of the substrate under study (inserted between the two tags), c) factors involved in the Endoplasmic Reticulum/Golgi/TransGolgi Network, d) factors involved in insertion or targeting to the Endoplasmic Reticulum, e) factors involved in the transport to the cell surface, f) cofactors required for any of the processes mentioned above or any of their combinations.

In alternative embodiments, exemplary assays of the invention are used as a platform for target discovery utilizing any molecular biology tool for their discovery. The assay can be easily coupled for target discovery with complementary DNA (cDNA) libraries, knockdown based technologies such as siRNA, shRNA, knock-in, knock out, overexpression of genes/proteins of interest, and others.

In alternative embodiments, exemplary assays of the invention are coupled to the event of cleavage itself. This exemplary assay, in contrast to others, pinpoints at the specific cells where the event of cleavage occurs. This is possible as the cleaved scaffold is not lost and it is detectable by one tag (HA as example). The assay is retrovirally engineered so one can back track and rescue any cell of interest within a population of cells.

The invention will be further described with reference to the following examples; however, it is to be understood that the invention is not limited to such examples.

### Examples

### Example 1: Exemplary Assays of the Invention

The invention provides compositions and assays for screening for inhibitors or modulators of enzymes or proteases, e.g., viral proteases such as HIV-1 protease (PR) (an aspartyl protease). PR is required for the efficient processing of the Gag and Gag-Pol precursor polyproteins; a critical step in the viral life cycle. In alternative embodiments, the invention provides compositions and assays for: (1) Discerning the effects of protease, e.g., PR, on signaling cascades of the host cell, and (2) Developing novel cell-based assays to enable screening of peptide libraries for the search of novel protease, e.g., PR, inhibitors or modulators. In alternative embodiments, a protease, e.g., PR, is expressed as a fusion protein in the presence of limiting levels of inhibitors or modulators, in different cellular compartments and in an inducible manner.

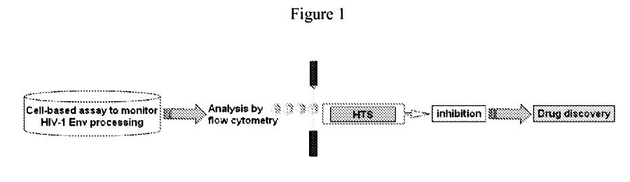

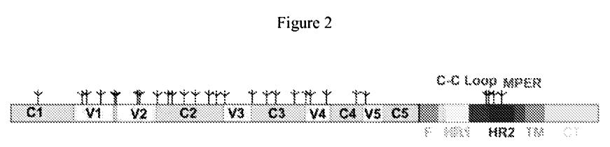

FIG. 1 schematically illustrates an exemplary cell-based drug discovery assay strategy of the invention. FIG. 2 schematically illustrates a gp160 Env precursor, which in alternative embodiments is incorporated into an exemplary scaffold protein of the invention. The gp160 Env precursor has: C1-C5 conserved regions and V1-V5 variable regions within gp120; the rest represents gp41: F, fusion peptide; HR1, heptad repeat 1; C—C loop, with the conserved disulfide bond; HR2, heptad repeat 2; MPER, membrane-proximal external region, TM, transmembrane domain; CT, cytoplasmic tail. Tree-like symbols represent glycans.

This invention provides a novel cell-based assay to provide insight into the mechanisms of HIV-1 envelope maturation. Envelope maturation, which occurs during classical transport through the Endoplasmic Reticulum-TransGolgi Network, is absolutely necessary for the production of infectious viral particles. Assays and compounds of this invention will facilitate the search for inhibitors/competitors of Envelope maturation, and as such will be the first of its kind, with huge impact in the fight against HIV-1.

In alternative embodiments, the invention provides cell-based assays that facilitate high throughput screening (HTS), in order to identify novel inhibitory compounds targeted against the production of infectious HIV-1 particles.

In alternative embodiments, the invention provides assays in an appropriate host cell context to screen for compounds that inhibit the recognition and/or cleavage of the gp120/gp41 boundary within the viral envelope. In alternative embodiments, fluorescence is utilized as the assay read-out, demonstrating its suitability for flow cytometry, and thus search for inhibitory compounds in a high throughput manner.

In alternative embodiments, the invention provides a rapid screening method for novel inhibitors of HIV-1 envelope processing, which is a crucial step in the production of infectious viral particles. In alternative embodiments, the invention provides an assay developed in the natural context, i.e. T-cells, and adaptable to flow cytometry, to enhance the utility and optimization of the assay for HTS, in turn drastically enhancing the chances of discovery of efficient compounds targeting HIV-1 envelope processing.

In alternative embodiments, the invention provides assays adapted to HTS platforms and in appropriate cellular contexts to facilitate identification of active and specific inhibitors of HIV-1 envelope processing. In alternative embodiments, the invention provides an assay that monitors HIV-1 envelope processing—recognition and/or cleavage of the gp120/gp41 boundary.

In alternative embodiments, the invention provides assays that specifically monitor the recognition and/or cleavage of the gp120/gp41 boundary within HIV-1 envelope. As such, these exemplary assays will greatly facilitate the discovery of a novel set of antivirals targeting envelope processing, with huge impact in the fight against HIV/AIDS.

We constructed a scaffold that can be used as the vector backbone for exemplary assays of the invention. In alternative embodiments, plasmids needed for the assays include an Lyt2/Env scaffold and relevant controls (with/out gp120/gp41 boundary and/or mutated boundary site). Their expected behavior can be corroborated in transient experiments at first and analyzed by flow cytometry.

In alternative embodiments, clonal cell lines are adapted for use in assays of the invention for HTS. Clones expressing the Lyt2/Env scaffold and relevant controls are selected and amplified. The utility of these clones can be ensured in e.g., 384- and 1536-well plate formats, and all parameters needed for HTS can be calibrated.

In alternative embodiments, assays of the invention screening with one or more libraries of chemical compounds, e.g., small molecules, e.g., using the NIH Molecular Libraries and Imaging Roadmap Initiative, or a chemical-compound library provided by the Molecular Libraries Production Centers Network to screen the selected clones, in order to e.g., identify potential novel inhibitory compounds targeted against the recognition and/or cleavage of the gp120/gp41 boundary.

In alternative embodiments, the invention provides cell-based assays in relevant host cell contexts. In alternative embodiments, assays of the invention monitor the activity of the viral protease in T-cells; the T-cell context provides the natural milieu necessary for HIV infection. In alternative embodiments, exemplary assays are engineered in T-cells. As gp160 processing occurs in infected cells, cell-based assays of this invention performed in an appropriate cell context will greatly enhance the study of HIV Env processing and maturation, a complex process still remaining to be fully elucidated. Moreover, as Env processing is critical for the production of infectious viral particles, Env processing and particularly Env recognition by Furin/PCs is an attractive target for antivirals. Exemplary assays can greatly enhance the discovery of a new kind of drugs specifically blocking the recognition and/or cleavage of the gp120/gp41 boundary, and assays of this invention can be a tool for their discovery. In alternative embodiments, a combination of combinatorial compound libraries and endogenously expressed random peptide libraries specifically targeted to the compartment where Env processing occurs are used, drastically increasing the chances of discovering novel drug candidates.

In alternative embodiments, cell-based assays are designed to specifically target Env processing, and can be suitable for HTS. Exemplary cell-based assays can monitor the cleavage of the gp120/gp41 boundary, greatly facilitating the search for HIV-1 Env processing inhibitors, which represents a completely novel kind of antivirals. Importantly, exemplary cell-based assays can facilitate the discovery of antivirals that inhibit the recognition/cleavage of Env rather than the activity of cellular enzymes, which would be probably detrimental to the cell. In alternative embodiments, cell-based assays are aimed at competitors for Env recognition rather than Furin inhibitors. In alternative embodiments, cell-based assays rely on the expression of a surface tag, and can be readily adaptable to a flow cytometry, enhancing its high throughput capabilities for drug discovery. In alternative embodiments, retroviral random peptide libraries are used for the screen of Env competitors for Furin/PCs recognition, which can be engineered to specifically localize to the ER/TGN luminal compartment.

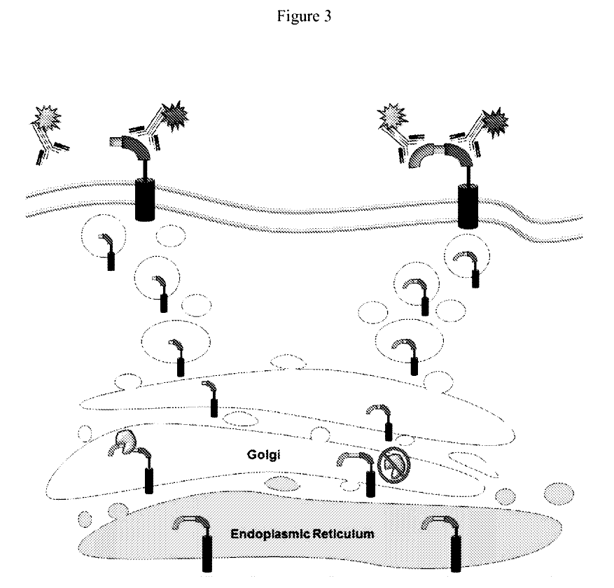

In alternative embodiments, cell-based assays of the invention comprise use of a double-tagged engineered scaffold (of the invention) that allows discrimination between cleaved and non-cleaved events based on the cell surface expression of one or two tags, respectively. In alternative embodiments the scaffold protein travels to the cell surface and retains the FLAG tag only when processing of the gp120/gp41 boundary within the gp160 Env precursor is inhibited, e.g., by a putative drug. In alternative embodiments, flow cytometry-based detection of the cell surface-expressed FLAG-tag serves as a biosensor for the gp160 boundary recognition and cleavage by Furin and related enzymes, i.e., cell surface FLAG expression directly correlates with blocked/inhibited gp120/gp41 boundary processing. A representation of an exemplary assay is depicted in FIG. 3: a scaffold protein containing two tags (HA and FLAG) separated by the gp120/41 Furin recognition/cleavage site, is targeted to the ER. Left: Furin cleaves the gp120/gp41 boundary, resulting in the loss of FLAG tag on the cell surface. Right: Furin is inhibited. Both HA and FLAG tags are recognized on the cell surface. Red rod: HA tag (closer to the extracellular surface), green Rod (distal from the cell surface): FLAG tag, yellow shape: Furin.

Env processing is critical for the production of infectious viral particles, rendering this process an attractive target for antivirals. In alternative embodiments, different gp120/gp41 Env boundaries with increasing sizes including different domains of gp41 are used and can be easily analyzed in the context of an assay of the invention. The robustness of the assay, together with its simplicity and the fact it is performed in the natural cellular milieu, all make the assays of the invention a perfect tool for elucidating the requirements for Env processing and trafficking, shedding light into one of the most complex processes in the HIV-1 life cycle. In alternative embodiments assays are engineered as an inducible retroviral system, ensuring stability of clones for long-term usage. These embodiments can be used for screening of a novel kind of drugs targeting the specific action of enzymes or proteases, e.g., Furin and similar PCs on Env processing.

In alternative embodiments, a combination of combinatorial compound libraries and random peptide libraries is expressed inside the cell and targeted to the ER-TGN compartment to drastically facilitate the discovery of drugs. In alternative embodiments the retrovirally delivered peptide libraries are expressed endogenously and are specifically engineered to be localized/targeted to the ER lumen. As each random peptide adopts a specific structure in space, it is expected that some peptides within the libraries (with expected complexities of several millions) to bear the right conformation needed to block the interaction between Env and Furin/PCs and/or cleavage. In alternative embodiments if/when a drug is found, peptidomimetics and biochemical studies are performed to further convert a putative peptide into a deliverable drug. In alternative embodiments assays of the invention are used as a platform for the study of processing of any viral envelope or cellular protein provided recognition and cleavage occurs in the ER-TGN.

Design of Lentiviral Constructs for the Expression of the Minimal Gp120/Gp41 Boundary Scaffold in T-Cells:

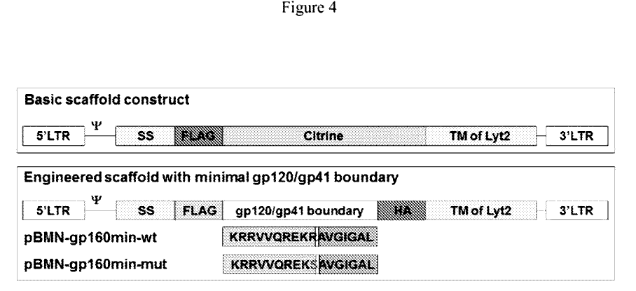

The murine CD8a homolog Lyt2 glycoprotein receptor was chosen as a scaffold for two reasons: First, its well-established topology and transport from the ER to the Golgi and trans-Golgi network (TGN) [53] for subsequent insertion into the outer cell membrane, and second, our extensive expertise with Lyt2-based engineering. We have exploited a construct we have previously developed where the green fluorescent protein citrine was fused to the Lyt2 TM at its C′ terminus and to the prolactin signal sequence fused to the FLAG tag at its N′ terminus, as illustrated in FIG. 4 (the so-called “basic scaffold construct”). FIG. 4 schematically illustrates exemplary constructs of the invention, or constructs that can be used to practice methods of the invention, including an exemplary engineered scaffold with a minimal gp120/gp41 boundary, including a pBMN-gp160 min-wt (SEQ ID NO:1) and pBMN-gp160 min-mut (SEQ ID NO:2):

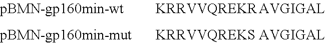

The prolactin signal sequence ensures proper insertion into the Endoplasmic Reticulum (ER) for transport to the cell surface while the FLAG tag ensures antibody-based recognition. For this exemplary assay, the citrine sequence of the basic scaffold construct (FIG. 4) was replaced by the gp120/gp41 boundary fused to the HA tag. As a result, the gp120/gp41 boundary is flanked by the FLAG tag at its N′ terminus and the HA tag at its C′ terminus, which is fused to the Lyt2 TM. This construct ensures that the Env segment faces the lumen of the ER/TGN. In this manner, if Furin/PCs recognize and cleave the gp120/gp41 boundary, the FLAG tag will be released. However, if blocked or inhibited, the FLAG tag will remain attached to the HA-tagged scaffold. The resulting retroviral construct is referred to as pBMN-gp160 min-wt for “minimal-wild-type gp120/gp41 boundary”. A construct with one point mutation substituting an arginine with a serine, resulting in a mutated non-cleavable recognition site and referred to as pBMN-160 min-mut (FIG. 4), is used as negative control.

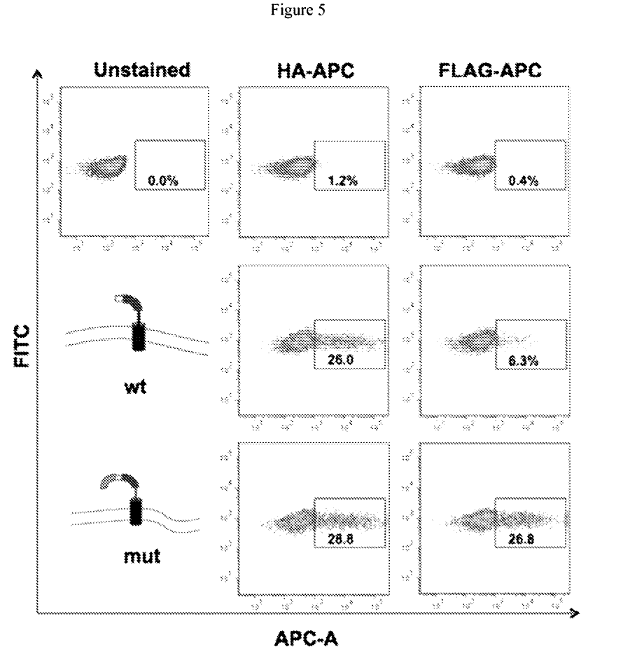

FIG. 5 illustrates graphically and schematically: transient expression in 293T cells of the pBMN-gp160 min-wt (SEQ ID NO:1)-comprising and pBMN-gp160 min-mut (SEQ ID NO:2)-comprising constructs; and, graphically illustrates a flow cytometry analysis from experiments using naive cells (top panels), or infected with wt gp120/gp41 boundary (mid panels) or mutant boundary (bottom panels) constructs, which were analyzed by flow cytometry post-transfection. APC-anti-HA and FITC-anti-FLAG antibodies were used for staining.

Transient Experiments Corroborate the Expected Results of the Engineered Assay:

While the assay is intended for stable expression in mammalian cells, we first analyzed the behavior of the engineered constructs in transient expression experiments. 293T cells transfected with pBMN-gp160 min-wt and pBMN-gp160 min-mut, were analyzed by flow cytometry following staining with APC-coupled HA antibodies, FITC-coupled FLAG antibodies or both. (In the basic citrine scaffold construct APC-coupled FLAG antibodies were used instead). The experiment demonstrates beyond any doubt that the mutated gp120/gp41 boundary is recognized by both HA and FLAG antibodies (FIG. 5). This proves two independent facts: First, the scaffold travels to the cell surface (HA positive), and second, that it is not cleaved (FLAG positive). The wt boundary, while traveling to the surface (HA positive), was cleaved, at least partially (drastic reduction of FLAG positive cells). The transient expression experiment, while demonstrating trend, is not intended to show robustness as transfection efficiency and protein expression level might not be optimal. (26-28% HA expression rather than close-to-100%, FIG. 5).

Selected Clones Corroborate Robustness of the Engineered Assay:

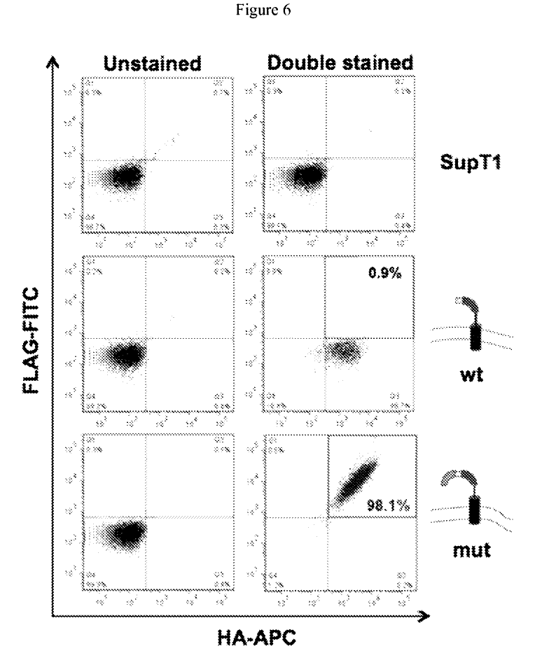

The transient expression experiments clearly demonstrated that the assay behaved as expected and reassured us to proceed with the stable expression experiments aimed at obtaining stable clones. Clonal populations should drastically increase robustness as 100% of the cells are expected to express the assay scaffold. For that purpose we utilized our extensive expertise with retroviral technology [54-56] in order to transfer the assay element into SupT1 T-cells for its stable expression. Selected clones sorted into a 96-well plate, were amplified for a period of one month and analyzed by flow cytometry and fluorescence microscopy. Flow cytometry analysis shows a high level of scaffold expression in both wild type (wt) and mutant gp120/gp41 boundary-expressing clones, as seen by approximately 100% staining in the HA-APC axis (FIG. 6). Importantly, only the mutant version shows approximately 100% double staining while the wt is completely lost. This proves the assay to be as robust as it can biologically be.

FIG. 6 illustrates graphically and schematically: transient expression in 293T cells of the pBMN-gp160 min-wt (SEQ ID NO:1)-comprising and pBMN-gp160 min-mut (SEQ ID NO:2)-comprising constructs; and, graphically illustrates a flow cytometry analysis of SupT1 clones stably expressing the assay. Naïve cells or cells expressing the wt or the mutant version of the boundary were analyzed following staining with both APC-coupled anti-HA and FITC-coupled anti FLAG antibodies.

Inhibited Wt Construct Further Corroborates the Robustness and Utility of the Assay:

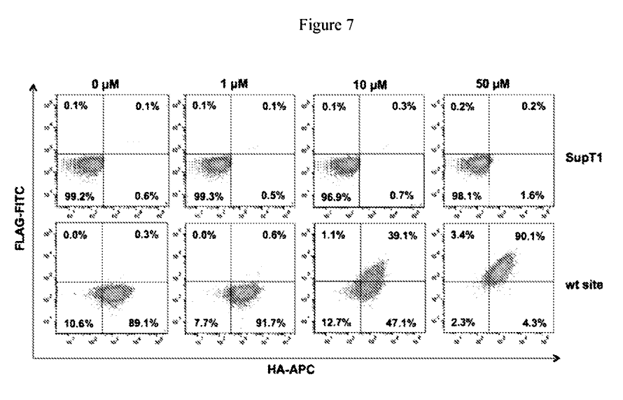

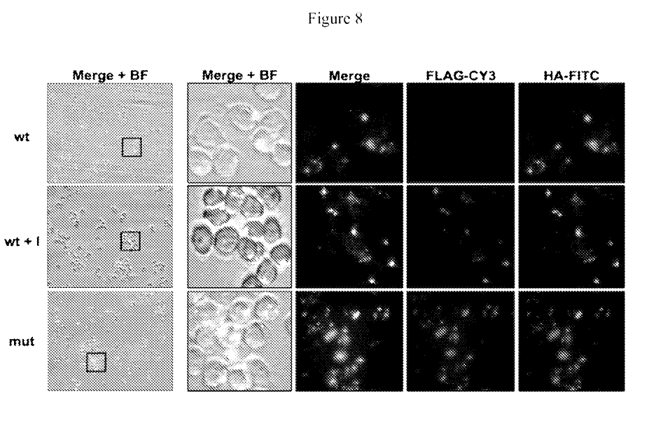

In an attempt to further demonstrate the robustness of the assay and its utility for future drug discovery it was important to prove whether an inhibitor can reverse the observed trend. For that purpose, clones expressing the wt gp120/41 boundary were analyzed by flow cytometry following incubation with increasing concentration of the Furin inhibitor DCK. FLAG surface expression is progressively recovered at 10 mM and 50 mM DCK, increasing form 0.9% to 39% and then 90% (FIG. 7). The same clones, treated with 50 mM DCK, were further analyzed by fluorescence microscopy prior and following incubation with 50 mM DCK. The mutant version was used as control. As seen in FIG. 8, while the clones expressing the mutant version are both green and red (yellow in the merge), clones expressing the wt version are only green. Importantly, when treated with DCK red fluorescence is restored (as is yellow). These results prove without a doubt the robustness of the assay and utility for screening.

FIG. 7 illustrates graphically and schematically: SupT1 clones stably expressing the assay (as with FIGS. 5 and 6, above) following inhibition with DCK. Clones were treated with increasing concentration of DCK and stained with FITC-coupled anti FLAG antibodies. Naïve cells were used as control for antibody staining.

FIG. 8 illustrates: Fluorescence microscopy images of SupT1 clones stably expressing the assay (as with FIGS. 5, 6 and 7, above). Clones expressing wild type (wt) or mutant version of the boundary were analyzed with HA-FITC and FLAG-CY3. Visible light, individual channels and merge are shown. I: Inhibitor, BF: Bright Field.

Tet-on System for Inducible Expression:

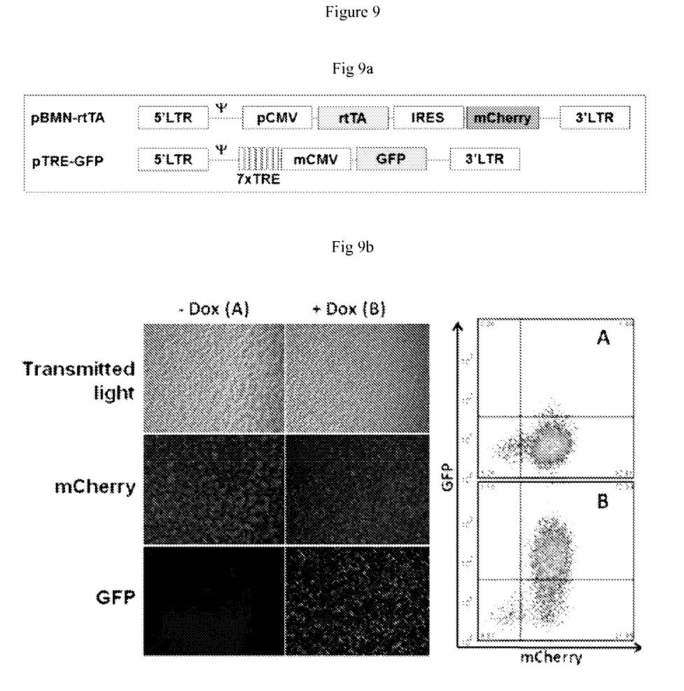

In alternative embodiments, constructs are expressed in an inducible manner to be able to turn on the expression of the scaffold proteins only when desired. As preliminary studies we have tested the tetracycline inducible system (Tet-On), adapted from Clontech. The system relies on the reverse tetracycline transactivator (rtTA), allowing induction of expression only upon addition of Tet or doxycycline (Dox). rtTA binds to the Tetracycline Responsive Element (TRE), which we have introduced in an HIV-based self-inactivating vector, with most of the 3′ HIV Long Terminal Repeat (LTR) U3 sequence deleted for safety reasons (FIG. 9). The system includes a retroviral vector carrying the rtTA element (pBMN-rtTA), coupled to an IRES-mCherry cassette (which will be replaced with a blasticidin resistance cassette for selection), and a lentiviral vector with an inducible promoter consisting of seven copies of TRE and a minimal Cytomegalovirus promoter (mCMV), driving the expression of the Green Fluorescence protein (GFP). A preliminary experiment was performed with SupT1 cells transduced with retroviral particles carrying pBMN-rtTA and pTRE-GFP to corroborate inducibility of the system. As seen in FIG. 9, cells fluoresce red as they express the mCherry protein constitutively (coupled to rtTA) but turn green only in the presence of Dox.

FIG. 9A illustrates schematically: exemplary retroviral/lentiviral constructs for inducible expression: the rtTA coupled to mCherry (the so called exemplary pBMN-rtTA construct) and the TRE coupled to a minimal CMV promoter driving GFP (the so called exemplary pTRE-GFP construct). FIG. 9B illustrates schematically and graphically results from studies using these constructs: left panels illustrate fluorescence microscopy images showing activation of GFP only in the presence of Doxycycline (Dox); and right panels graphically represent these images by flow cytometry. (LTR: Long Terminal Repeat, Y: packaging signal, IRES: Internal Ribosome Entry Site).

### Research Design and Methods

Assay Overview:

In order to establish a reliable and reproducible cell-based assay to monitor the recognition/cleavage of the gp120/gp41 Env boundary, facilitate the study of Env maturation and maximize throughput capabilities, the following points are addressed: Stable expression of the assay elements using retroviral vectors and selection of clones; Adaptation to inducible expression to avoid the possible toxic side effects of an ER-TGN-targeted protein; Adaptation to other strains of HIV; Establishment of the assay in T-cells, a cell-type that mimics the natural environment of HIV infection; Adaptation of the assay to 96 and 384-well formats facilitating HTS. Studies demonstrate beyond any doubt the robustness of the assay for the assessment of cleavage by Furin/PCs, at least of the minimal gp120/gp41 HIV-1 Env boundary.

Adaptation to an Inducible System:

As continuous expression of the scaffold may overwhelm the ER transport machinery and have cytotoxic effects, they can be expressed in an inducible manner via the Tet-On system, e.g., as adapted from Clontech. This will allow to turn expression on only when needed, upon addition of Dox. The fusion protein of the original vector [51] can be replaced with a Lyt2/Env scaffold and transferred into an rtTA-expressing SupT1 cell line. Since these are retroviral vectors, they enable the stable expression of the desired proteins, which optimizes clone selection.

Corroboration of Reproducibility and Stability of the Assay Over Time:

In order to ensure the reproducibility of the assay over time and the stability and utility of the clonal cell populations for long term usage, chosen clones can be analyzed over a period of time, throughout which they will be treated with Dox, stained and analyzed by flow cytometry at two-week intervals. As the constructs used are retroviral/lentiviral in nature, routinely used for the expression of ectopic information for months to years, this assures that the clones will be stable and functional for long periods of time.

Implementation of the Assay in 96 and 384-Well Format:

In alternative embodiments, the assay is intended for drug discovery, so it can be important to calibrate the system in large-number-of-well plate format. In alternative embodiment, the assay uses cell populations in 96-well to a 384-well or 1536-well plate format. The sample volume acquired by flow in an HTS set-up represents 10-20% of the total working volume of the well; 4-to-8 μl for a 384-well plate. Thus around 4,000-to-8,000 cells can be plated per well in the latter, enabling a concentration of about 100-to-200 cells/μl, sufficient for staining and flow cytometry analysis. Exact conditions for proper staining and washing in 96 and 384-well plates can be defined. In order to ensure reproducibility across the plate, experiments can be done in triplicates; three 384-well plates can be loaded with 5,000-10,000 cells/well and treated with 1 μg/mL Dox, with/out peptidyl inhibitor (when needed). Plates can then be stained with fluorophore-coupled anti-FLAG antibody and analyzed by flow cytometry to corroborate that fluorescence is similar across the entire plate and across different plates.

Analysis of Env Elements Required for Furin Cleavage:

In alternative embodiments, the largest portion of gp160 that still allows recognition and cleavage by Furin/PCs in the context of the assay is used, with the goal of mimicking wild-type conformation. The HXB2 T-tropic wild-type HIV-1 sequence can be used as consensus to engineer our constructs. In alternative embodiments, a minimal gp120/gp41 boundary is used, or an increasing number of motifs known to be important for Furin recognition and tertiary structure can be used. In alternative embodiments the goal is to mimic, as much as possible, the natural requirements for cleavage. In alternative embodiments, for each construct, a version with the mutated cleavage site is included. Their expected behavior can be corroborated in transient experiments; and also can be transferred to SupT1 through viral transduction for the selection of clones.

Construction of Env-Based Proteins:

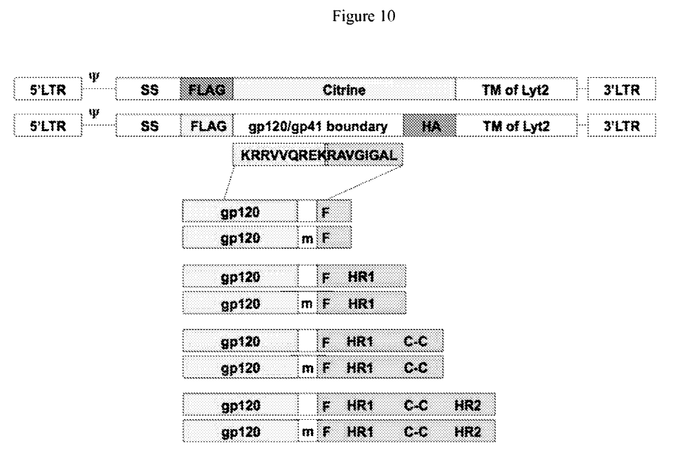

In alternative embodiments, several versions of the gp120/gp41 boundary are used in order to calibrate and ensure the utility and robustness of the assay. All the outlined constructs can be amplified by PCR from the same template and mutated versions will be constructed by site-directed mutagenesis. For each of the constructs both wild type (wt) and mutated sites (indicated as “m”) can be included (FIG. 10, constructs a and b, respectively). While the constructs described below include an increasing number of motifs within the gp41 portion of gp160, larger portions of gp120, particularly C3 through C5 (see FIG. 1), believed to be important for the three dimensional structure and contact with gp41 motifs (REFs), which may influence Furin/PCs recognition, can be used.

FIG. 10 illustrates: Constructs with increasing length of the gp120/gp41 boundary. Olive boxes represent sequences within gp120 and blue boxes within gp41. F: fusion peptide, HR1 and 2: N-heptad repeat 1 and 2, m: mutated site, C—C: disulfide bond, m: mutated cleavage site.

FIG. 10A: Minimal gp120/gp41 boundary (‘Preliminary Data’).
FIG. 10B: Boundary with the gp41 fusion peptide (F).
FIG. 10C: Boundary, wild type cleavage site (wt).
FIG. 10D: Boundary, with mutated cleavage site (indicated as “m”).
FIG. 10E: Boundary with the N-heptad repeat 1 (HR1), wild type (wt) cleavage site.
FIG. 10F: Boundary with the N-heptad repeat 1 (HR1), with mutated cleavage site.
FIG. 10G: Boundary with HR1 and disulfide bond (C—C), wild type (wt) cleavage site.
FIG. 10H: Boundary with HR1 and disulfide bond (C—C), with mutated site cleavage site.
FIG. 10I: Boundary with HR1, C—C and HR2, wild type (wt) cleavage site.
FIG. 10J: Boundary with HR1, C—C and HR2, with mutated site cleavage site.

Also illustrated in FIG. 10 is the exemplary the 120/gp41 “boundary”, or cleavage site, having the sequence (SEQ ID NO:3) KRRVVQREKRAVGIGAL.

Analysis of the Expression Pattern:

Once the recombinant lentiviral vectors have been generated, transient transfection experiments with HEK293T adherent cells can corroborate their expression (Western blot), specifically, their expression on the cell surface (flow cytometry). To verify intracellular expression and localization confocal microscopy analysis with fluorescence-coupled anti-FLAG antibodies can be performed. As performed with the minimal region, wild-type with mutated versions of the scaffolds can be compared, as well as wild-type with and without the commercially available DCK inhibitor (Calbiochem, shown to bind to the catalytic site of Furin and block its activity [45]).

The minimal construct can be used as control as has been proven to travel to the surface and lose the FLAG tag unless inhibited or mutated, as illustrated in FIG. 10A (the first construct listed). Expression of the entire gp160 molecule can be cumbersome, and the anchoring domain can be deleted, as illustrated in FIG. 10B. Exemplary constructs FIG. 10C and FIG. 10D, can demonstrate whether F retains the construct or improves recognition and cleavage. Same rationale follows for exemplary constructs 10E through 10J, where additional motifs are added. Exemplary constructs FIG. 10E and FIG. 10F, can demonstrate whether the N-heptad repeat HR1, which facilitates trimerization [11, 31], is needed for processing in the context of our assay. Exemplary constructs FIG. 10G and FIG. 10H, can show how addition of the C—C disulfide bond, shown to interact with gp120 C5 for proper conformation and recognition by Furin/PCs [32], affects the assay. For exemplary constructs FIG. 10G and FIG. 10H, the C—C disulfide bond and gp120/gp41 boundary are distant sequence-wise. Constructs FIG. 10I and FIG. 10J contain the entire Env sequence flanking the region between the gp120 C5 domain and HR2; this construct can allow proper conformation and trimerization, and thus Furin/PC cleavage. For each construct the overall expression of HA on the surface can be compared to the minimal construct to define whether transport was partially or completely hindered.

Establishment of Clonal Cell Lines Expressing Individual Scaffold Proteins:

The lentiviral constructs shown to behave as expected in transient experiments can be transduced through lentiviral particles into the rtTA-expressing SupT1 cell line. Three days post-transduction, individual cells are sorted into individual wells of a 96-well-plate, and amplified. Dox can be added to induce scaffold expression and cell surface expression analyzed by flow cytometry following anti-FLAG antibody staining. Clones can be chosen based on highest cell-surface expression relative to their cell-surface expression when treated with DCK and/or compared to their mutated counterparts will be chosen, expanded and frozen in aliquots for future use.

Screening for the Discovery of Novel Drugs Against Gp160 Processing:

In alternative embodiments, assays of the invention can screen a variety of libraries, e.g., a combinatorial chemical library (e.g., as the Torrey Pines institute for Molecular Studies (TPIMS) combinatorial chemical library) as well as the retroviral peptide libraries. This screen can be performed with the minimal gp120/gp41 boundary (Aim 1) and/or with any of the larger fragments.

Libraries for Screening:

In alternative embodiments different libraries are screened, increasing the chances of finding putative new drugs. In alternative embodiments, each screen is independent of each other and can be done independently. In alternative embodiments, libraries include:

1. The Combinatorial Library of TPIMS:

TPIMS libraries include more than five million small molecule compounds assembled as systematically arranged mixtures of approximately 2,000 compounds each in average. Arranged in 96-well plates, they will be de-convoluted at different stages during the screening process based on positive FLAG staining. This approach allows the assay to produce hits without first biasing the screening collection.

2. Retroviral ER-TGN-Retained Random Peptide Library:

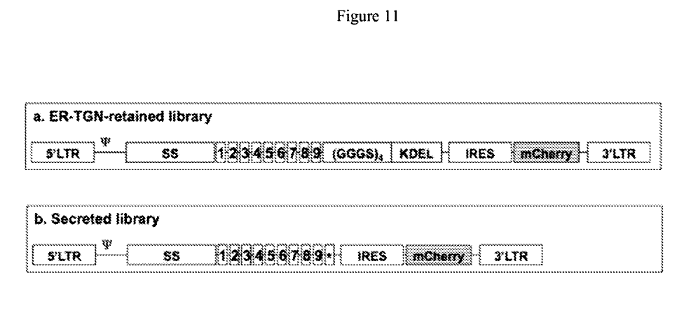

For the expression of a random peptide library localized to the ER/TGN luminal compartment, a retroviral vector carrying a protein scaffold composed of a signal sequence, the site for library insertion, a long flexible link and a KDEL ER retention signal (construct a in FIG. 11a) is engineered. The well-established signal sequence of prolactin can be used for targeting engineered proteins to the ER. In alternative embodiments, after being cleaved by Furin and other PCs in the ER/TGN lumen, the mature protein is allowed to travel to the cell surface through the classical secretion pathway. In alternative embodiments, a KDEL motif, a well-established ER retention signal through KDEL receptors (REF), is incorporated at the C-terminal tail of the scaffold; it can ensure the protein to be retained and/or recycled back into the luminal compartment when Env processing occurs.

In alternative embodiments, a long flexible link, composed of four repetitions of the Glycine/Glycine/Glycine/Serine motif, between the random peptide library and the KDEL motif, is used to ensure relative freedom of movement and flexibility within the ER/TGN lumen, most probably required to interfere with Furin/PC recognition and/or cleavage of HIV Env. The rationale behind the construction of a library localized to the ER/TGN luminal compartment is obviously to increase the chances of physical interaction between the players involved in Env processing. Moreover, the retroviral nature of the library ensures the continuous expression of the peptide fusion required for optimal competition. As the retroviral plasmid carries an IRES-mCherry cassette, cells expressing a peptide will fluoresce red.

3. Retroviral Secreted Random Peptide Library:

In alternative embodiments, for the expression of a secreted random peptide library, a random sequence is introduced into a vector carrying a protein scaffold very similar to the one described for the ER-TGN-retained library. In alternative embodiments, the scaffold will not contain the flexible link or the KDEL retention signal and will be allowed to travel to the cell surface. The engineered retroviral vector can carry a protein scaffold composed of a signal sequence, the site for library insertion, and a stop codon (construct b in FIG. 11b). As it does not contain a TM, it can then be secreted into the medium. Here, the rationale behind the construction of a secreted library is, again, to increase the chances of interaction. Despite the fact that the peptides will be ultimately secreted, they will use the classical pathway for transport and thus be concomitantly present with Env and Furin/PCs. While each peptide might be present in the lumen for a short period of time, the retroviral constitutive expression will ensure a continuous source of peptide.

FIG. 11 illustrates: Constructs for the expression of the random peptide libraries. Scaffolds for: FIG. 11a. ER-TGN-retained peptides (the “ER-TGN retained library”), and, FIG. 11b. secreted peptides (the “secreted library”). (LTR: Long Terminal Repeat SS: Prolactin signal sequence, Y: packaging signal, IRES: Internal Ribosome Entry Site).

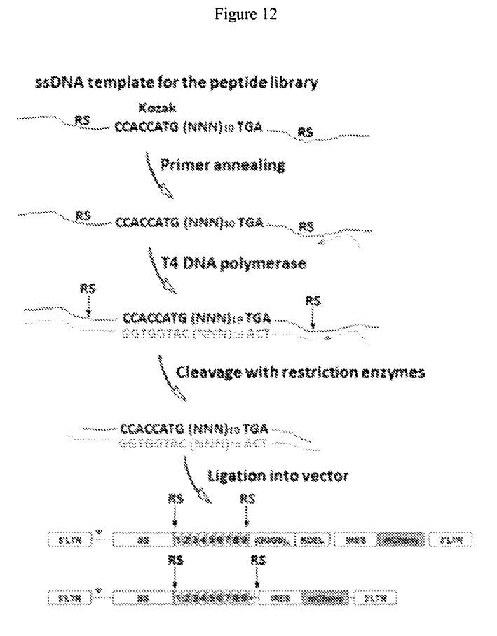

FIG. 12 illustrates an exemplary process of the invention for the construction of an exemplary peptide library, and also illustrates exemplary constructs of the invention. The process starts with the engineering of the ssDNA template carrying the degenerative EP67 peptide sequences and ends with the insertion of the library into the target retroviral vector. Once the dsDNA library is synthesized, it will be inserted into retroviral vectors for both intracellular and secreted peptides. Illustrated in FIG. 12 are: RS: Restriction site, (LTR: Long Terminal Repeat; Y: packaging signal; RS: Restriction sites; IRES: Internal Ribosome Entry Site; Kozak consensus sequence is CCACCATG (NNN)₁₀ TGA (SEQ ID NO:4), and its complementary sequence GGTGGTAC (NNN)₁₀ ACT (SEQ ID NO:5); GGGS peptide fragment (SEQ ID NO:6); and, KDEL (SEQ ID NO:7) (KDEL when this sequence is in an amino acid structure of a protein, it keeps the protein from being secreted from the endoplasmic reticulum (ER); it can also target proteins from other locations (such as the cytoplasm) to the ER; proteins can only leave the ER after this sequence has been cleaved off).

Transfer of the Peptide Libraries into Clones Carrying the Assay:

The peptide libraries carried by the retroviral vectors can be transferred into the selected SupT1 clonal populations expressing the assay. For the production of retroviral particles packaging cell lines can be transfected with the library-carrying retroviral vectors. The resulting viral particles can be collected and used to transduce target cells. As the peptide libraries are coupled to the IRES-mCherry cassette, cells expressing a peptide can become red fluorescence. Transduction can be performed in a very low multiplicity of infection so cells will be infected by a single viral particle to facilitate the propagation of clonal populations when putative hits are encountered. For that purpose at least 300 million cells can be infected at an expected infection rate of 10%. Following a period of two-three weeks, red fluorescent cells can be sorted out and amplified for further screening.

The Screening Process:

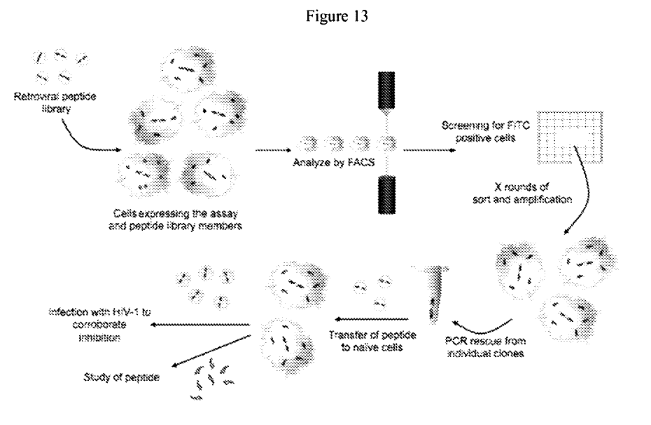

For the screening of the TPIMS libraries, cells harboring the assay alone can be screened. Cells can be transferred into pre-set library 96-well plates, which contain around 200 compounds per well. Following induction with Dox, and staining with FITC-coupled anti-FLAG antibody based on the conditions discussed above, the plate can be analyzed by flow cytometry. Wells showing any FLAG staining above background can be further de-convoluted. The process can be repeated until reaching plates with one compound per well, provided FLAG positive stain is observed along the way. For the screening of the peptide libraries, the cells carrying both the assay and the libraries can be used. An outline of the screening process is shown in FIG. 13. Basically, cells induced with Dox to express the assay and constitutively expressing peptides form the library (red cells) can be analyzed by flow cytometry. FITC positive cells (supposedly retaining FLAG on their surface) can be sorted out. The process of staining, sorting and amplification can be repeated as many times as needed to obtain a population of cells containing only FLAG-positive cells. Cells can then be individually plated into 96-well plates and amplified. A s peptides putatively responsible for FLAG retention are of retroviral nature, their sequence can be rescued by PCR amplification of the rescued clones.

Corroboration of Hits:

As schematically illustrated in FIG. 13, putative rescued peptides can be transferred into assay-bearing naïve cells to corroborate inhibition of cleavage and thus FLAG retention. Naïve cells incubated with putative compounds rescued from the combinatorial library and clones expressing putative peptides from the retroviral libraries can be infected with wt HIV-1. Their supernatant can be collected and used to re-infect naïve cells, which can be used to analyze the infection rate. Untreated naïve cells can be used as comparative control. Biochemical assays can be performed where the cleavage of the assay fusion protein can be analyzed by western blot to determine size in the presence or absence of putative compounds, or performed with cellular extracts from clones expressing the putative rescued peptides. Other secondary and tertiary assays can be done as needed.

In alternative embodiments, a HXB2 T-tropic wild-type HIV-1 sequence is used, but the assay can use other strains or clades of HIV. While the Furin recognition site is very conserved among the different strains, amino acid substitutions are found within gp120 and gp41 motifs known to influence the three-D structure of Env, and thus the recognition by Furin/PCs. The assays of the invention thus can be adapted to HIV-1 and HIV-2 as well as other clades of HIV-1 making it into a platform for the study of Env processing and drug discovery against all HIV viruses spread around the world. Assays of the invention can be adapted to other viruses that rely on processing in the ER-TGN compartment, such as members of the Flaviviridae. If putative hits obtained from the random peptide-library-based screen is not intended to find the final drug product and peptidomimetics and biochemical studies can be performed to further convert a putative peptide into a deliverable drug.

Production of Retroviral Particles:

For the production of MLV-based viral particles, Phoenix-GP packaging cell line (kindly provided by Garry Nolan, Stanford University, CA) was transfected with retroviral vectors. For the production of HIV-based virus particles, 293T cells were transfected with pH-GFP transfer vector, pCI-VSVg, and pCMV.Δ8.2 (Didier Trono, EPFL, Switzerland). In each case, viral supernatant was collected at 48 hours post-transfection. Viral supernatant was used to transduce SupT1 cells by centrifugation at 1500×g, at 32° C. for 80 minutes. Cells harboring rtTA were further transduced with viral particles carrying the wt gp120/gp41 boundary (pBMN-gp160 min-wt), or the mutant (pBMN-gp160 min-mut).

Flow cytometry and sorting: Flow cytometry is performed on a BD FACSAria™ and/or FACSCanto (SDSU FACS core facility) with 488 nm and 633 nm lasers. Data is collected using FACSDiva 6.1.1™ software (BD Biosciences, San Jose, Calif.) and analyzed by FLOWJO™ (Tree Star, Inc., Ashland, Oreg.).

### High Throughput and Multiplexed Adaptations

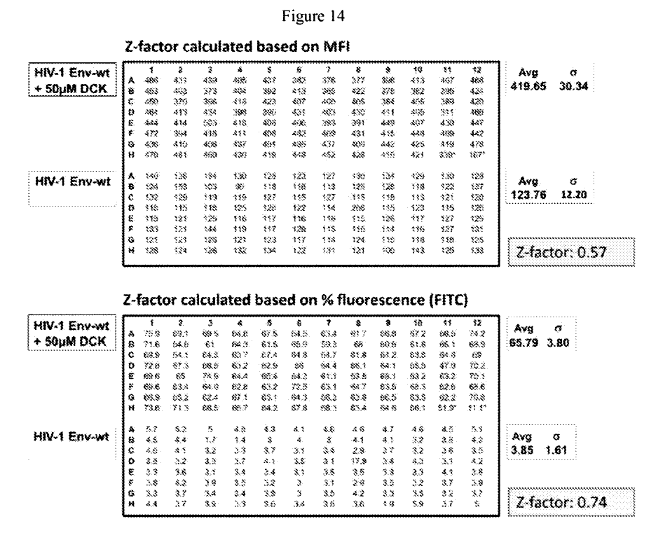

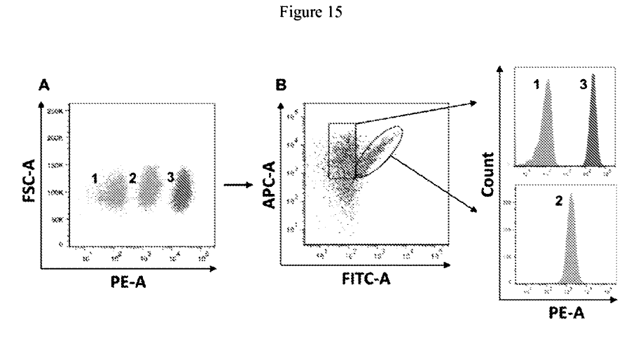

In alternative embodiments, assays of the invention can be adapted to any high-number well format for high content, high throughput adaptations. FIG. 14 schematically illustrates an analysis of an exemplary assay of the invention in a 96-well format. The high Z′ or Z-factor (0.57 based on MFI or 0.74 based on % fluorescence) demonstrates the assay's robustness and repeatability for high-throughput screening and other applications.

In alternative embodiments, multiplexing can be used to enhance high-throughput capabilities. The assay, in a multiplexed format, can be used as a platform that allows the analysis or investigation of multiple targets/substrates supposedly cleaved or recognized within the secretory pathway, in one sample. The assay can be coupled with barcoding, genetic or other, to increase multiplexing capabilities. Genetic fluorescent barcoding through retroviral technology, for example, allows multiplexing of the assay without further manipulations.

As example, FIG. 15 schematically illustrates a fluorescent flow cytometry analysis of three SupT1 T cell lines, each carrying a different substrate within the context of the assay elements. The distinct cell lines were previously genetically barcoded with different levels of tdTomato fluorescent protein. In that manner, the assay cells, when analyzed for tdTomato in the PE channel (FIG. 15A, panel A) show three populations (negative, dim, and bright tdTomato). In contrast, when analyzed for the assay in the two other channels (FIG. 15B), one tag (HA in the APC channel) or two tags (HA in the APC channel and FLAG in the FITC channel) only two populations are revealed (panel B below). Importantly, the HA positive/FLAG negative population actually reveals two populations when analyzed for tdTomato: They include Population 1 (negative for tdTomato expressing the HIV envelope wt boundary) and Population 3 (bright for tdTomato expressing the DenV pr-M boundary). The HA positive/FLAG positive population reveal Population 2 (dim for tdTomato expressing the mutant HIV envelope boundary; env-mut (R511S)). In that manner, multiplexing through genetic barcoding allowed to deconvolute or decode the analysis, revealing a larger number of populations, each expressing a different substrate.

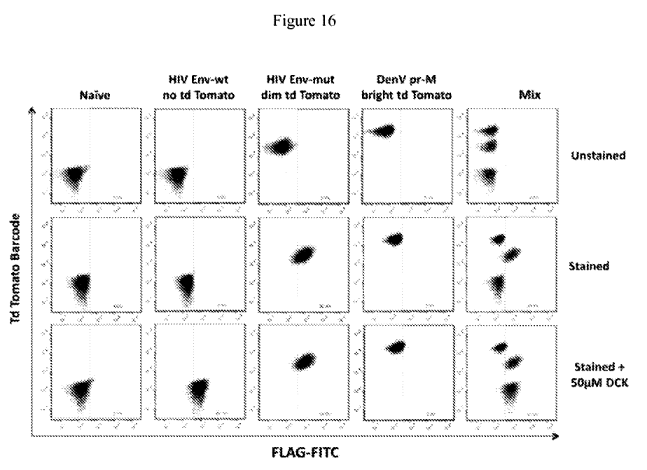

In order to further prove the adaptability of exemplary assays of the invention to a multiplexed format, the same three populations where analyzed by flow cytometry independently or mixed in the same sample. FIG. 16 schematically illustrates a fluorescent flow cytometry analysis in the PE channel for td Tomato expression and FITC channel for FLAG expression. As a reminder, in the assay the second tag (FLAG here) is lost if cleavage occurs while the scaffold protein travels to the cell surface. In the last column of panels the mixed sample reveals the three populations based on tdTomato expression. When stained for FLAG, only population 2 (mutant HIV-1 boundary) retains the tag, while wild type HIV-1 boundary and DenV pr-M, as expected, lost it.

In alternative embodiments, exemplary assays of the invention are used for drug discovery. As a proof of principle, the same experiment with the three bar-coded populations was repeated in the presence of DCK, an inhibitor of Furin and similar protein convertases that are active in the Golgi/TransGolgi network. DCK treatment reconstitutes the second tag (FLAG) cell surface expression only with wildtype HIV1-envelope boundary, proving that Furin or similar enzymes are responsible for the cleavage of the HIV-1 envelope boundary. Interestingly, while DenV pr-M was expected to be cleaved by Furin, it was not inhibited by DCK, proving that this exemplary assay can pinpoint specificity of substrate recognition or specificity of enzyme involved in cleavage.

In alternative embodiments, exemplary assays of the invention are used for pinpointing substrate and/or enzyme specificity with the use of drugs or other compounds. Interestingly, while DenV pr-M was expected to be cleaved by Furin, it was not inhibited by DCK, proving that this exemplary assay can pinpoint specificity of substrate recognition or specificity of enzyme involved in cleavage.

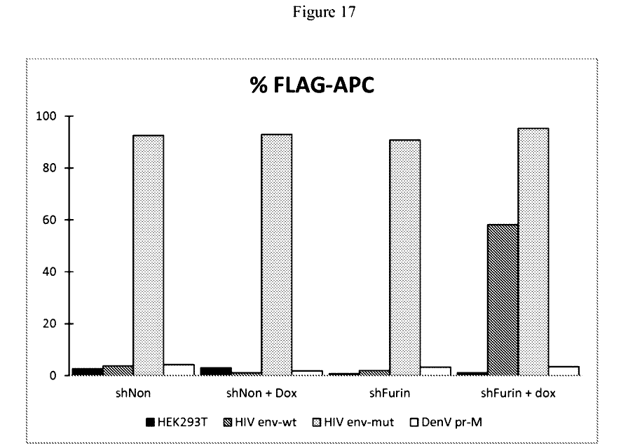

In alternative embodiments, exemplary assays of the invention are used for pinpointing substrate, enzyme specificity, and/or requirement of factors with the use of drugs and/or RNA-knockdown-based techniques or similar. The assay can be couple to RNAi technologies for example to pinpoint specificity of protease cleavage of substrates and elucidate unknown targets, proteases, and/or cleavage events. FIG. 17 graphically illustrates experiment data demonstrating that inducible short interfering RNA expression against Furin corroborates the results obtained and shown in FIG. 16. In this experiment Doxycycline (Dox) is used to induce shRNA expression. Expression of shRNA against Furin reconstitutes FLAG expression only of wildtype HIV-1 envelope boundary but not Deny pr-M. This proves that that this exemplary assay can be used to assess specificity of cleavage and pinpoint the requirement or lack off of specific enzymes in cleavage within the secretory pathway, e.g., as in this case, Furin.

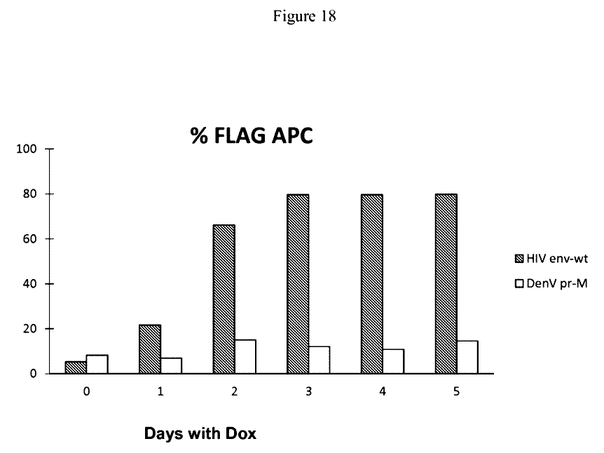

Knockdown of Furin reconstitutes FLAG expression with wildtype HIV-1 envelope boundary for a period of at least five days, as graphically illustrated in FIG. 18, while no reconstitution is observed with DenV-prM for the same period, further corroborating this exemplary assay can be used to study specificity of cleavage and/or recognition. Knockdown of Furin was further demonstrated by quantitative PCR.

This exemplary assay's versatility can be further proved as it can be easily adapted to any substrate of interest, viral or host. To prove versatility of the assay, other viral substrates were analyzed. The substrates that were included in the flow cytometry analysis are:

The WNV pr-M wt substrate is: CTKTRHSRRSRRSLTVQTHG (SEQ ID NO:8)

The DenV pr-M wt substrate is: CTTTGEHRREKRSVALVPHV (SEQ ID NO:9)

The DenV pr-M mut substrate is: CTTTGEHRREKSVALVPHV (SEQ ID NO:10)

The HIV Env wt substrate is: KRRVVQREKRAVGIGAL (SEQ ID NO:11)

The HIV Env mut substrate is: KRRVVQREKSAVGIGAL (SEQ ID NO:12)

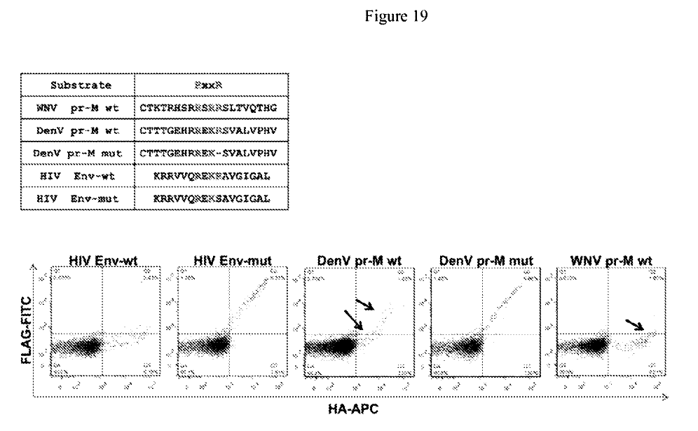

This includes not only HIV wild type and mutant boundaries, and wild type DenV pr-M boundary, but also a mutant DenV pr-M boundary as additional control and the pr-M boundary of West Nile virus. As shown in the flow cytometry data schematically illustrated in FIG. 19, the pattern of cleavage is different in the different examples further demonstrating that this exemplary assay can be used for studying the specific requirements for recognition and/or cleavage of the different substrates/targets of interest.

In alternative embodiments, exemplary assays of the invention are adapted to other viral substrates that utilize the classical secretory pathway. HIV-1 utilizes the pathway for the transport of its envelope protein to the cell surface and in its way it is cleaved by Furin and similar protein convertases. All viruses of the Flaviviridae family are tightly associated with the Endoplasmic Reticulum and/or the Golgi TransGolgi network.

While we have already adapted the assay to HIV-1 envelope and to Dengue virus and West Nile virus pr-M boundaries, the assay can be adapted to other viral segments that serve as putative substrates of enzymes that are active in the classical secretory pathway. These include viruses or all viral families that exploit the secretory pathway, such as Flaviviridae, which include, but are not restricted to: Dengue virus, Hepatitis C Virus, West Nile virus, Yellow fever virus, Japanese encephalitis virus, Tick-borne encephalitis virus, Kyasanur Forest disease virus, Murray Valley encephalitis virus, St. Louis encephalitis virus, bovine viral diarrhoea virus, Rio Bravo virus, Culex flavivirus or pegivirus. Other examples include viral proteins from other families, some of which are listed in the table of FIG. 20, including influenza, papilloma, Sindbis and Ebola:

HIV-1 gp 160
(SEQ ID NO: 13)
PTKAKRRVVQREKRAVGIGA
HIV-2 gp140
(SEQ ID NO: 14)
PVKRYSSAPVRNKRGVFVLG
Dengue Virus pr-M
(SEQ ID NO: 15)
GTCTTTGEHRREKRSVALVP
Influenza A Virus HA
(SEQ ID NO: 16)
ATGPRNVPQRRKKRGLFGAK
Human Papilloma Virus 16 L2
(SEQ ID NO: 17)
MRHKRSAKRTKRASATQL
Ebola Virus (Zaire) GP
(SEQ ID NO: 18)
GVAGLITGGRRTRREAIVNA
Shigella dysentariae Shiga Toxin subunit A
(SEQ ID NO: 19)
LILNCHHHASRVARMASDEF
Bacillus anthracis Anthrax Toxin Protective
Antigen
(SEQ ID NO: 20)
PELKQKSSNSRKKRSTSAGP
Corynebacterium diphtheria Diptheria Toxin
(SEQ ID NO: 21)
YMAQACAGNRVRRSVGSSL
Pseudomona aeroginosa exotoxin A
(SEQ ID NO: 22)
HLPLETFTRHRQPRGWEQLE
Human 13 Amyloid Precursor Cleaving Enzyme (BACE1)
(SEQ ID NO: 23)
SGLGGAPLGLRLPRETDEEP
Human Matrix Metalloproteinase 2 (MMP2)
(SEQ ID NO: 24)
DLDQNTIETMRKPRCGNPDV
Human Insulin-like Growth Factor 1 (IGF1)
(SEQ ID NO: 25)
LKPAKSARSVRAQRHTDMPK
Human Transforming Growth Factor 13 (TGF β)
(SEQ ID NO: 26)
YRLESQQTNRRKKRALDAAY
Human Vascular Endothelial Growth Factor C
(VEGF C)
(SEQ ID NO: 27)
KLDVYRQVHSIIRRSLPATL

### REFERENCES CITED

1. Frey, G., et al., A fusion-intermediate state of HIV-1 gp41 targeted by broadly neutralizing antibodies. Proc Natl Acad Sci USA, 2008. 105(10): p. 3739-44.
2. HIV vaccine failure prompts Merck to halt trial. Nature, 2007. 449(7161): p. 390.
3. Hammer, S. M., et al., Antiretroviral treatment of adult HIV infection: 2008 recommendations of the International AIDS Society-USA panel. Jama, 2008. 300(5): p. 555-70.
4. Bradac, J. and C. W. Dieffenbach, HIV vaccine development: Lessons from the past, informing the future. IDrugs, 2009. 12(7): p. 435-9.
5. Pettit, S. C., et al., Processing sites in the human immunodeficiency virus type 1 (HIV-1) Gag-Pro-Pol precursor are cleaved by the viral protease at different rates. Retrovirology, 2005. 2: p. 66.
6. Speck, R. R., et al., Comparison of human immunodeficiency virus type 1 Pr55(Gag) and Pr160(Gag-pol) processing intermediates that accumulate in primary and transformed cells treated with peptidic and nonpeptidic protease inhibitors. Antimicrob Agents Chemother, 2000. 44(5): p. 1397-403.
7. Chan, D. C. and P. S. Kim, HIV entry and its inhibition. Cell, 1998. 93(5): p. 681-4.
8. Gallo, S. A., et al., The HIV Env-mediated fusion reaction. Biochim Biophys Acta, 2003. 1614(1): p. 36-50.
9. Baker, K. A., et al., Structural basis for paramyxovirus-mediated membrane fusion. Mol Cell, 1999. 3(3): p. 309-19.
10. Caffrey, M., et al., Three-dimensional solution structure of the 44 kDa ectodomain of SIV gp41. Embo J, 1998. 17(16): p. 4572-84.
11. Chan, D. C., et al., Core structure of gp41 from the HIV envelope glycoprotein. Cell, 1997. 89(2): p. 263-73.
12. Eckert, D. M. and P. S. Kim, Mechanisms of viral membrane fusion and its inhibition. Annu Rev Biochem, 2001. 70: p. 777-810.
13. Joshi, S. B., R. E. Dutch, and R. A. Lamb, A core trimer of the paramyxovirus fusion protein: parallels to influenza virus hemagglutinin and HIV-1 gp41. Virology, 1998. 248(1): p. 20-34.
14. Malashkevich, V. N., M. Singh, and P. S. Kim, The trimer-of-hairpins motif in membrane fusion: Visna virus. Proc Natl Acad Sci USA, 2001. 98(15): p. 8502-6.
15. Skehel, J. J. and D. C. Wiley, Receptor binding and membrane fusion in virus entry: the influenza hemagglutinin. Annu Rev Biochem, 2000. 69: p. 531-69.
16. Weissenhorn, W., et al., Structural basis for membrane fusion by enveloped viruses. Mol Membr Biol, 1999. 16(1): p. 3-9.
17. Weissenhorn, W., et al., Atomic structure of the ectodomain from HIV-1 gp41. Nature, 1997. 387(6631): p. 426-30.
18. Yu, M., et al., Six-helix bundle assembly and characterization of heptad repeat regions from the F protein of Newcastle disease virus. J Gen Virol, 2002. 83(Pt 3): p. 623-9.
19. Decroly, E., et al., The convertases furin and PC1 can both cleave the human immunodeficiency virus (HIV)-1 envelope glycoprotein gp160 into gp120 (HIV-1 SU) and gp41 (HIV-I TM). J Biol Chem, 1994. 269(16): p. 12240-7.
20. Earl, P. L., B. Moss, and R. W. Doms, Folding, interaction with GRP78-BiP, assembly, and transport of the human immunodeficiency virus type 1 envelope protein. J Virol, 1991. 65(4): p. 2047-55.
21. Freed, E. O. and M. A. Martin, The role of human immunodeficiency virus type 1 envelope glycoproteins in virus infection. J Biol Chem, 1995. 270(41): p. 23883-6.
22. Hallenberger, S., et al., The role of eukaryotic subtilisin-like endoproteases for the activation of human immunodeficiency virus glycoproteins in natural host cells. J Virol, 1997. 71(2): p. 1036-45.
23. Henderson, L. E., et al., Quantitative separation of murine leukemia virus proteins by reversed-phase high-pressure liquid chromatography reveals newly described gag and env cleavage products. J Virol, 1984. 52(2): p. 492-500.
24. Kamps, C. A., Y. C. Lin, and P. K. Wong, Oligomerization and transport of the envelope protein of Moloney murine leukemia virus-TB and of is 1, a neurovirulent temperature-sensitive mutant of MoMuLV-TB. Virology, 1991. 184(2): p. 687-94.
25. Moulard, M. and E. Decroly, Maturation of HIV envelope glycoprotein precursors by cellular endoproteases. Biochim Biophys Acta, 2000. 1469(3): p. 121-32.
26. Moulard, M., et al., Processing and routage of HIV glycoproteins by furin to the cell surface. Virus Res, 1999. 60(1): p. 55-65.
27. Doms, R. W., et al., Folding and assembly of viral membrane proteins. Virology, 1993. 193(2): p. 545-62.
28. Hallenberger, S., et al., Inhibition of furin-mediated cleavage activation of HIV-1 glycoprotein gp160. Nature, 1992. 360(6402): p. 358-61.
29. McCune, J. M., et al., Endoproteolytic cleavage of gp160 is required for the activation of human immunodeficiency virus. Cell, 1988. 53(1): p. 55-67.
30. Si, Z., et al., Small-molecule inhibitors of HIV-1 entry block receptor-induced conformational changes in the viral envelope glycoproteins. Proc Natl Acad Sci USA, 2004. 101(14): p. 5036-41.
31. Fass, D., S. C. Harrison, and P. S. Kim, Retrovirus envelope domain at 1.7 angstrom resolution. Nat Struct Biol, 1996. 3(5): p. 465-9.
32. Sen, J., et al., The disulfide loop of gp41 is critical to the furin recognition site of HIV gp160. Protein Sci, 2007. 16(6): p. 1236-41.
33. Binley, J. M., et al., A recombinant human immunodeficiency virus type 1 envelope glycoprotein complex stabilized by an intermolecular disulfide bond between the gp120 and gp41 subunits is an antigenic mimic of the trimeric virion-associated structure. J Virol, 2000. 74(2): p. 627-43.
34. Cao, J., et al., Effects of amino acid changes in the extracellular domain of the human immunodeficiency virus type 1 gp41 envelope glycoprotein. J Virol, 1993. 67(5): p. 2747-55.
35. Jacobs, A., et al., Alanine scanning mutants of the HIV gp41 loop. J Biol Chem, 2005. 280(29): p. 27284-8.
36. Maerz, A. L., et al., Functional analysis of the disulfide-bonded loop/chain reversal region of human immunodeficiency virus type 1 gp41 reveals a critical role in gp120-gp41 association. J Virol, 2001. 75(14): p. 6635-44.
37. York, J. and J. H. Nunberg, Role of hydrophobic residues in the central ectodomain of gp41 in maintaining the association between human immunodeficiency virus type 1 envelope glycoprotein subunits gp120 and gp41. J Virol, 2004. 78(9): p. 4921-6.
38. Caffrey, M., Model for the structure of the HIV gp41 ectodomain: insight into the intermolecular interactions of the gp41 loop. Biochim Biophys Acta, 2001. 1536(2-3): p. 116-22.
39. Malashkevich, V. N., et al., Crystal structure of the simian immunodeficiency virus (SW) gp41 core: conserved helical interactions underlie the broad inhibitory activity of gp41 peptides. Proc Natl Acad Sci USA, 1998. 95(16): p. 9134-9.
40. Moulard, M., et al., Retroviral envelope glycoprotein processing: structural investigation of the cleavage site. Biochemistry, 1998. 37(13): p. 4510-7.
41. Ugolini, S., et al., HIV-1 gp120 induces an association between CD4 and the chemokine receptor CXCR4. J Immunol, 1997. 159(6): p. 3000-8.
42. Liu, S., et al., Rapid and automated fluorescence-linked immunosorbent assay for high-throughput screening of HIV-1 fusion inhibitors targeting gp41. J Biomol Screen, 2003. 8(6): p. 685-93.
43. Ou, W., Y. Xiong, and J. Silver, Quantification of virus-envelope-mediated cell fusion using a tetracycline transcriptional transactivator: fusion does not correlate with syncytium formation. Virology, 2004. 324(2): p. 263-72.
44. Coppola, J. M., et al., Identification of inhibitors using a cell-based assay for monitoring Golgi-resident protease activity. Anal Biochem, 2007. 364(1): p. 19-29.
45. Wu, C., et al., Furin-mediated processing of Pro-C-type natriuretic peptide. J Biol Chem, 2003. 278(28): p. 25847-52.
46. San Jose, E., M. A. Munoz-Fernandez, and B. Alarcon, Megalomicin inhibits HIV-1 replication and interferes with gp160 processing. Virology, 1997. 239(2): p. 303-14.
47. Matthews, T., et al., Enfuvirtide: the first therapy to inhibit the entry of HIV-1 into host CD4 lymphocytes. Nat Rev Drug Discov, 2004. 3(3): p. 215-25.
48. Bewley, C. A., et al., Design of a novel peptide inhibitor of HIV fusion that disrupts the internal trimeric coiled-coil of gp41. J Biol Chem, 2002. 277(16): p. 14238-45.
49. He, Y., et al., Peptides trap the human immunodeficiency virus type 1 envelope glycoprotein fusion intermediate at two sites. J Virol, 2003. 77(3): p. 1666-71.
50. Ou, W. and J. Silver, Inhibition of murine leukemia virus envelope protein (env) processing by intracellular expression of the env N-terminal heptad repeat region. J Virol, 2005. 79(8): p. 4782-92.
51. Hilton, B. J. and R. Wolkowicz, An assay to monitor HIV-1 protease activity for the identification of novel inhibitors in T-cells. PLoS One, 2010. 5(6): p. e10940.
52. Rajakuberan, C., B. J. Hilton, and R. Wolkowicz, Protocol for a Mammalian Cell-Based Assay for Monitoring the HIV-1 Protease Activity. Methods Mol Biol, 2012. 903: p. 393-405.
53. Dong, C. and G. Wu, Regulation of anterograde transport of adrenergic and angiotensin II receptors by Rab2 and Rab6 GTPases. Cell Signal, 2007. 19(11): p. 2388-99.
54. Wolkowicz, R., G. C. Jager, and G. P. Nolan, A random peptide library fused to CCRS for selection of mimetopes expressed on the mammalian cell surface via retroviral vectors. J Biol Chem, 2005. 280(15): p. 15195-201.
55. Wolkowicz, R. and G. P. Nolan, Retroviral technology—applications for expressed peptide libraries. Front Biosci, 2003. 8: p. d603-19.
56. Wolkowicz, R., G. P. Nolan, and M. A. Curran, Lentiviral vectors for the delivery of DNA into mammalian cells. Methods Mol Biol, 2004. 246: p. 391-411.
57. Okumura, Y., et al., The extracellular processing of HIV-1 envelope glycoprotein gp160 by human plasmin. FEBS Lett, 1999. 442(1): p. 39-42.

A number of embodiments of the invention have been described. Nevertheless, it will be understood that various modifications may be made without departing from the spirit and scope of the invention. Accordingly, other embodiments are within the scope of the following claims.

1. A chimeric or recombinant scaffold protein comprising:
(i) an amino acid motif or subsequence susceptible to cleavage by an enzyme or a protease under physiologic or cell culture conditions,
(ii) a transmembrane domain;
(iii) a signal sequence or any amino acid motif that places the scaffold protein on the extracellular surface of the cell, or a signal sequence for scaffold protein insertion into the Endoplasmic Reticulum (ER) for transport to the cell surface,
wherein optionally the signal sequence is a prolactin signal sequence; and
(iv) at least two detectable moieties,
wherein: when the scaffold protein is expressed on the cell surface the amino acid motif or subsequence susceptible to cleavage by the enzyme or protease is positioned within the scaffold protein such that at least one of the detectable moieties is distal to the cleavable target motif with respect to the cell,
and at least one of the detectable moieties is between the cleavable target motif and the cell surface when the scaffold protein is expressed extracellularly,
thus, if the enzyme or protease is active in the cell, the detectable moiety or moieties distal to the cleavable target motif will not be present or part of the scaffold protein expressed on the cell surface,
but when an effective amount of an inhibitor or a competitor to the enzyme or protease is present in the cell, the cleavable target motif is not cleaved and the detectable moiety or moieties distal to the cleavable target motif will be present or part of the scaffold protein expressed on the cell surface.

2. An isolated, recombinant or synthetic nucleic acid encoding the scaffold protein of claim 1,
wherein the nucleic acid is operatively linked to a transcriptional regulatory unit, and optionally the transcriptional regulatory unit comprises a promoter, and optionally the promoter is a constitutive or an inducible promoter.

3. A vector, expression cassette, cosmid or plasmid comprising or having contained therein the isolated, recombinant or synthetic nucleic acid of claim 2.

4. A cell comprising:
(a) the chimeric or recombinant scaffold protein of claim 1;
(b) the cell of (a), wherein the enzyme or protease is heterologous to the cell and the cell further comprises a heterologous nucleic acid encoding the heterologous enzyme or protease,
and optionally the enzyme or protease-coding sequence is operatively linked to the same or a similar transcriptional regulatory unit as the nucleic acid encoding the scaffold protein of claim 1;
(c) the cell of (a) or (b), wherein the cell constitutively or inducibly expresses the chimeric or recombinant scaffold protein; or
(d) the cell any of (a) to (c), wherein the cell is a mammalian cell, a monkey cell, or a human cell, or a lymphocyte or a T-cell.

5. A cell line derived from the cell of claim 4.

6. A non-human transgenic animal comprising: the chimeric or recombinant scaffold protein of claim 1.

7. A cell-based method for monitoring the activity of an enzyme, a protease, a viral protease, or an HIV-1 protease (PR), comprising:
(a) providing a cell or cell line that expresses the chimeric or recombinant scaffold protein of claim 1,
wherein optionally the enzyme or protease is endogenous to the cell or is heterologous to the cell; and
(b) determining whether the detectable moiety or moieties distal to the cleavable target motif on the scaffold protein is expressed on the scaffold protein on the extracellular surface of the cell, or whether or not the detectable moiety or moieties distal to the cleavable target motif on the scaffold protein are or are not be present or part of the scaffold protein expressed on the cell surface,
wherein lack of detection of the detectable moiety or moieties distal to the cleavable target motif on the scaffold protein indicates that the enzyme or protease is active in the cell, and detection of the detectable moiety or moieties distal to the cleavable target motif on the scaffold protein indicates the presence of an inhibitor or a competitor of the enzyme or protease,
wherein extracellular detection of the detectable moiety or moieties positioned on the scaffold protein between the cleavable target motif and the cell surface when the scaffold protein is expressed on the cell surface indicates or confirms that the scaffold protein is expressed on the cell surface.

8. The cell-based method of claim 7, further comprising screening for a putative inhibitor or competitor of an enzyme, a protease, a viral protease, an HIV protease, or an HIV-1 protease, by:
(a) providing a compound to be screened as an inhibitor or a competitor of an enzyme, a protease, a viral protease, an HIV protease, or an HIV-1 protease; or a nucleic acid encoding a protein to be screened as an inhibitor or a competitor of an enzyme, a protease, a viral protease, an HIV protease, or an HIV-1 protease;
(b) contacting a plurality of the cells with the compound or nucleic acid of (a), wherein optionally the contacting is either before, during and/or after expression of the scaffold protein-expressing nucleic acid in the cell,
and optionally the nucleic acid encoding a protein to be screened as an inhibitor or a competitor of an enzyme, a protease, a viral protease, an HIV protease, or an HIV-1 protease is expressed before, during and/or after expression of the scaffold protein-expressing nucleic acid in the cell; and
(c) determining whether the detectable moiety or moieties distal to the cleavable target motif on the scaffold protein is expressed on the scaffold protein on the extracellular surface of the cell, or whether or not the detectable moiety or moieties distal to the cleavable target motif on the scaffold protein are or are not be present or part of the scaffold protein expressed on the cell surface,
wherein lack of detection of the detectable moiety or moieties distal to the cleavable target motif on the scaffold protein indicates that the enzyme or protease is active in the cell, and detection of the detectable moiety or moieties distal to the cleavable target motif on the scaffold protein indicates that the compound is acting as an inhibitor or a competitor of the enzyme or protease,
wherein extracellular detection of the detectable moiety or moieties positioned on the scaffold protein between the cleavable target motif and the cell surface when the scaffold protein is expressed on the cell surface indicates or confirms that the scaffold protein is expressed on the cell surface.

9. The cell-based method of claim 8, further comprising running a negative control comprising dividing the plurality of the cells and not adding the compound to be screened as an inhibitor or competitor to one of the divided cell samples, or not expressing the putative inhibitor or competitor in one of the divided cell samples.

10. The cell-based method of claim 7, further comprising running a positive control comprising dividing the plurality of the cells and adding a compound known to be an inhibitor or competitor of the enzyme or protease to one of the divided cell samples, or expressing a known inhibitor or competitor of the enzyme or protease in one of the divided cell samples.

11. The cell-based method of claim 7, formatted for multiplexed or high-throughput screening of compounds of test compounds or drugs.

12. The method of claim 7, wherein the detectable moiety is detected or measured on the extracellular surface of the cell by a high throughput screen (HTS), a flow cytometry or a microscope visualization.

13. The method of claim 7, wherein the compound to be screened as an inhibitor of the enzyme, protease, viral protease or HIV-1 protease comprises a small molecule, a nucleic acid, a polypeptide or peptide, a peptidomimetic, a polysaccharide or a lipid.

14. The method of claim 7, wherein the compound to be screened as an inhibitor of the enzyme, protease, viral protease or HIV-1 protease is a member of a library of compounds to be screened, or a member of a random peptide library or a chemical compound.

15. The method of claim 7, wherein the compound to be screened comprises a plurality of compound comprising or from a combination of: retroviral random peptide libraries; combinatorial compound libraries; and/or, endogenously expressed random peptide libraries specifically targeted to the compartment where Env processing occurs.

16. The chimeric or recombinant scaffold protein of claim 1, wherein:
(a) the scaffold protein comprises all of, substantially all of, or part of: a murine CD8a homolog or a Lyt2 transmembrane domain glycoprotein receptor polypeptide as the scaffold;
(b) the enzyme or protease is a viral enzyme or protease, or an HIV enzyme or protease, or a furin, or a proteinase or peptidase; or
(c) the amino acid motif or subsequence susceptible to cleavage is from or is derived from a virus of the family Flaviviridae, or a Dengue virus, a Hepatitis C Virus, a West Nile virus, a Yellow fever virus, a Japanese encephalitis virus, a Tick-borne encephalitis virus, a Kyasanur Forest disease virus, a Murray Valley encephalitis virus, a St. Louis encephalitis virus, a bovine viral diarrhoea virus, a Rio Bravo virus, a Culex flavivirus or pegivirus, an influenza virus, a papilloma virus, a Sindbis virus and/or an Ebola virus; or, an amino acid motif or subsequence susceptible to cleavage as illustrated in FIG. 20.

17. The chimeric or recombinant scaffold protein of claim 1, wherein:
(a) the enzyme or protease is a viral, a bacterial, an Archaeal, a eukaryotic, a mammalian, a human, an HIV or an HIV-1 enzyme or protease, or a furin;
(b) the cleavable target motif is a viral, a bacterial, an Archaeal, a eukaryotic, a mammalian, or a human sequence, optionally within an HIV-1 envelope (Env) protein, and optionally the cleavable target motif is cleaved by a furin or a protein convertase; or
(c) the cleavable target motif is at the gp120/41 boundary within a gp160 Env poly-protein.

18. The chimeric or recombinant scaffold protein of claim 1, wherein:
(a) one of the detectable moieties is a FLAG tag or an epitope for an antibody,
(b) the chimeric or recombinant scaffold protein of (a), wherein the FLAG tag is positioned distal to the cleavable target motif with respect to the cell when the scaffold protein is on the extracellular surface of the cell;
(c) one of the detectable moieties is a Human influenza hemagglutinin (HA) tag; or
(d) chimeric or recombinant scaffold protein of (c), wherein the HA tag is positioned on the scaffold protein between the cleavable target motif and the extracellular surface of the cell when the scaffold protein is on the extracellular surface of the cell.
